(19) United States
(12) Patent Application Publication
(10) Pub. No.: US 2021/0005278 A1
(43) Pub. Date: Jan. 7, 2021

(54) SYSTEMS AND METHODS FOR PREDICTIVE NETWORK MODELING FOR COMPUTATIONAL SYSTEMS, BIOLOGY AND DRUG TARGET DISCOVERY

(71) Applicants: ARIZONA BOARD OF REGENTS ON BEHALF OF THE UNIVERSITY OF ARIZONA, Tucson, AZ (US); Mount Sinai School of Medicine, New York, NY (US)

(72) Inventors: Rui Chang, Tucson, AZ (US); Eric Schadt, New York, NY (US)

(21) Appl. No.: 16/976,421

(22) PCT Filed: Feb. 27, 2019
(86) PCT No.: PCT/US2019/019864

§ 371 Date: Aug. 27, 2020

Related U.S. Application Data
(60) Provisional application No. 62/635,946, filed on Feb. 27, 2018.

(51) Int. Cl. G16B 5/20; G16B 30/00; G16B 40/00; G06N 7/00; G06N 20/00

(52) U.S. Cl. CPC G16B 5/20; G16B 30/00; G06N 20/00; G06N 7/005; G16B 40/00

(57) ABSTRACT

Systems and methods for predictive network modeling are disclosed. The systems and methods disclosed compute a top-down causal model and a bottom-up predictive model and utilize those models to determine the conditional independence among multiple variables and causality among equivalent variable structures. Before or during modeling, the data is passed through Markov Chain Monte Carlo sampling.

### CROSS-REFERENCE TO RELATED APPLICATIONS

This application claims the benefit of U.S. Provisional App. No. 62/635,946, filed on Feb. 27, 2018, the entire contents of which are hereby incorporated by reference.

### GOVERNMENT LICENSE RIGHTS

This invention was made with government support under Grant Nos. NIA 1 RF1 AG057457-01 and NIH/NIDDK P30DK116074 awarded by the National Institute of Health. The government has certain rights to the invention.

### FIELD OF INVENTION

The present invention relates to computer-implemented systems and methods related to predictive network modeling or top-down & bottom-up predictive network modeling. More specifically, the present invention relates to predictive network modeling for applications in computer networks, the biological sciences, and in drug target discovery.

### BACKGROUND OF THE RELATED ART

The problem of inferring causality between variables, especially recovering causal networks from observation data is a particularly challenging task. Bayesian network is one state-of-the-art approach aiming to recover the conditional independence among a set of variables from observation. However, it is well known that Bayesian networks fail to distinguish causal structures in equivalent classes because these structures are comprised of multiple chains of pairwise variable encode having the same joint probability and conditional independence.

The nonlinear nature between variables and systematic noise in observation data provide an important clue to infer causality direction. The direction of the causality is inferred as the hypothesis producing the least residuals in response to the fluctuation in data.

### SUMMARY OF THE INVENTION

It is therefore an object of the invention to disclose a holistic framework, which integrates a Bayesian network learning (top-down) algorithm with recently developed causality inference using an integrated Qualitative Knowledge-based Bayesian Inference (QKBI) (bottom-up) method. In previous works, it has been shown that given a correct causality configuration of a biology network topology, QKBI can yield accurate predictions on the marginal probability of variables in the network. Therefore, the bottom-up QKBI approach will directly evaluate the hypothesis on causality by predicting marginal probabilities on current causal structure and compare it to the observation data. Because predicted marginal probability is sensitive to the causality direction in network, the proposed method enhanced Bayesian network learning to infer not only conditional independence but also causality.

The proposed approach leverages a piece-wise constrained linear regression model in a transformed signal space as a good approximation to the full nonparametric model, also referred to as the Bernard-nonparametric model. One of the more prominent advantages of the systems and methods disclosed herein is that unlike the complex nonparametric model, constrained linear regression provides intuitive and effective representation of the causal assumptions in the model and enables us to derive a closed-form expression for the residual of causality model fitting. Secondly, previously derived constraints over conditional probability distribution reconcile the Bottom-up probabilistic inference in a Bayesian framework with the causality inference problem in continuous signal space. This allows us to integrate the bottom-up inference method with the state-of-the-art Bayesian network structure learning (top-down) and embed the causality inference in a pair-wise and equivalent structure class into a causal network structure learning problem. Thirdly, unlike previously known optimization techniques, in which a single evaluation value is derived and used to estimate the causality hypothesis, the integrated approach disclosed herein leverages the full Bayesian concept which does not take any single ‘best’ fit for granted, to infer the direction and function of the causality. The fitting residuals are calculated from a set of models in constrained subspace. Unlike other fitting methods to observation data, the systems and methods herein rely on a set of constraints over the model parameters to perform the fitting by sampling each possible parameter from the restricted sub-region in the model parameter space and generate predictions (marginal probability inference) in each model sample. Final fitness is measured by distance between the averaged prediction from all constrained models and the observation data. The employment of constraints relieved the systems and methods from heavy dependence on the observation data and prevents overfitting to the data, especially when it is sparse.

Insulin resistance (IR) precedes the development of type 2 diabetes (T2D) and increases cardiovascular disease risk. Although genome wide association studies (GWAS) have uncovered new loci associated with T2D, their contribution to explain the mechanisms leading to decreased insulin sensitivity has been very limited. Thus, new approaches are necessary to explore the genetic architecture of insulin resistance. To that end, an iPSC library was generated across the spectrum of insulin sensitivity in humans. RNA-seq based analysis of 310 induced pluripotent stem cell (iPSC) clones derived from 100 individuals allowed us to identify differentially expressed genes and pathways between insulin resistant and sensitive iPSC lines. Analysis of the co-expression architecture uncovered several insulin sensitivity-relevant gene sub-networks, and predictive network modeling identified a set of key driver genes that regulate these co-expression modules. Functional validation in human adipocytes and skeletal muscle cells (SKMCs) confirmed the relevance of the key driver candidate genes to insulin responsiveness.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is an exemplary embodiment of the invention, showing the hardware used in its implementation;

FIG. 2A shows an exemplarily Bayesian network where every variable is conditionally independent of its non-descendants given its parents; Three causal structures from the same equivalent structure class become undistinguishable by Bayesian network.

FIG. 2B shows an exemplary partial directed graph of a typical Bayesian network as a result of undistinguishable equivalent structures.

FIG. 2C exemplarily shows Bayesian network structure can be further improved by predictive network learning method as the network score from Bayesian network learning is further improved after continued with predictive network learning;

FIG. 2D exemplarily shows the improvement over standard Bayesian techniques in the precision and recall using the algorithms of the present invention;

FIG. 3 is an exemplary logic flow diagram demonstrating how the invention predictively reaches causal inferences;

FIG. 4 shows a data analysis pipeline followed by software in an exemplary application of the algorithm related to Alzheimer's drug targets;

FIG. 5 shows the results from an exemplary application of the algorithm as it relates to Abeta40 and Abeta42 results for RGS4;

FIG. 6 shows the results from an exemplary application of the algorithm as it relates to Aβ results for PDHB.

FIG. 7 shows a flowchart detailing the individual steps of the integrative predictive network modeling analysis pipeline and functional molecular validation: sample collection, data generation, data normalization, differential expression analysis, co-expression networks, predictive networks and key driver analysis, prioritization of KDs and molecular and functional validation;

FIG. 8A shows a differential expression analysis for All-Samples (AS) where 1338 differentially expressed genes with multiple testing corrected p-value<0.05 are shown. The color scale indicates normalized residual expression levels (blue: low expression, orange: high expression), and the columns have been arranged according to samples/donors, (purple: insulin resistant, turquoise: insulin sensitive);

FIG. 8B shows a differential expression analysis for Average-per-Patient (ApP): top 500 differentially expressed genes identified ApP are shown. The color scale indicates normalized residual expression levels (blue: low expression, orange: high expression), and the columns have been arranged according to samples/donors, (purple: insulin resistant, turquoise: insulin sensitive);

FIG. 9A shows the topological overlap matrix (TOM) of insulin resistance (IR) in AS. The topological overlap matrix (TOM) indicates co-expression between genes and their corresponding pathway enrichment analysis (PEA) of the IR and insulin sensitive (IS) co-expression network in AS and ApP, and color bars in each TOM indicate different co-expression modules. Only genes included in co-expression modules are shown. Genes included in the grey co-expression module (containing genes that could not be assigned to any other co-expression modules) are not shown;

FIG. 9B shows the TOM of IS in AS. The TOM indicates co-expression between genes and their corresponding PEA of the IR and IS co-expression network in AS and ApP, and color bars in each TOM indicate different co-expression modules. Only genes included in co-expression modules are shown. Genes included in the grey co-expression module (containing genes that could not be assigned to any other co-expression modules) are not shown;

FIG. 9C shows the TOM of IR in ApP. The TOM indicates co-expression between genes and their corresponding PEA of the IR and IS co-expression network in AS and ApP, and color bars in each TOM indicate different co-expression modules. Only genes included in co-expression modules are shown. Genes included in the grey co-expression module (containing genes that could not be assigned to any other co-expression modules) are not shown;

FIG. 9D shows the TOM of IS in ApP. The TOM indicates co-expression between genes and their corresponding PEA of the IR and IS co-expression network in AS and ApP, and color bars in each TOM indicate different co-expression modules. Only genes included in co-expression modules are shown. Genes included in the grey co-expression module (containing genes that could not be assigned to any other co-expression modules) are not shown;

FIG. 9E shows the corresponding PEA results for the co-expression network shown in FIG. 9A;

FIG. 9F shows the corresponding PEA results for the co-expression network shown in FIG. 9B;

FIG. 9G shows the corresponding PEA results for the co-expression network shown in FIG. 9C;

FIG. 9H shows the corresponding PEA results for the co-expression network shown in FIG. 9D;

FIG. 10A shows a predictive causal network (PCN) that elucidates the key drivers and sub-networks of the tested key drivers for, from left to right, the PCN of IS in ApP, IR in ApP, IS in AS, and IR in AS;

FIG. 10B shows a predictive causal network (PCN) that elucidates the key drivers and sub-networks of the tested key drivers for the key drivers replicated in all of the networks;

FIG. 10C shows a predictive causal network (PCN) that elucidates the key drivers and sub-networks of the tested key drivers for the sub-network of key drivers from left to right corresponding to the order of PCN in FIG. 10A;

FIG. 11A shows the differential pathway enrichment after HMGCR inhibition in IR vs IS iPSCs as a Venn diagram for the IR-specific (442 genes), IS-specific (367 genes) and IR/IS overlapping (343 genes) DE genes due to HMGCR inhibition;

FIG. 11B shows the pathway enrichments for the groups defined in FIG. 11A. The top-10 significant pathways are shown for each group;

FIG. 12A shows the Predictive Network Validation (AS network) by RNA-seq data in Atorvastatin treated iPSC lines, where the percentage of DE genes decreases as the distance (layers) from HMGCR increases;

FIG. 12B shows the Predictive Network Validation (AS network) by RNA-seq data in Atorvastatin treated iPSC lines, where, among the genes located downstream of HMGCR, the fold change decreases as the distance to HMGCR increases;

FIG. 12C shows the Predictive Network Validation (AS network) by RNA-seq data in Atorvastatin treated iPSC lines, where the p-value of differential expression analysis from the Atorvastatin experiment decreases along the steps down from HMGCR. The upper lane is AS IR network, while the lower lane is the AS IS network;

FIG. 13A shows the Predictive Network Validation by RNA-seq data among the genes located downstream of HMGCR in both the IR and IS networks in the ApP network;

FIG. 13B shows another Predictive Network Validation by RNA-seq data among the genes located downstream of HMGCR in both the IR and IS networks in the ApP network

FIG. 14A shows the functional validation of key driver genes, where the insulin mediated glucose uptake assay in mature human adipocytes. Fold change values are shown with respect to no insulin (−). Each panel shows the effect of varying concentrations of inhibitor for HMGCR (atorvastatin), FDPS (alendronate) and SQLE (terbinafine);

FIG. 14B shows the functional validation of key driver genes, where an adipogenic differentiation assay is shown. The effect of different concentrations of the inhibitors over SGBS adipogenesis is measured by absorbance measurement of Oil-O-Red emission at 500 nM;

FIG. 14C shows the functional validation of key driver genes, where a growth assay is performed in human SGBS preadipocytes. Crystal violet staining is performed after 12 days of assay in presence/absence of the inhibitors;

FIG. 14D shows the functional validation of key driver genes, where an insulin mediated glucose uptake assay is performed in mature human SKMCs; and

FIG. 14E shows the functional validation of key driver genes, where a growth assay in human SKMCs. Results represent mean±SD, and statistical significance was evaluated through One-way ANOVA *p<0.05, **p<0.01, ***p<0.001 compared to insulin condition without inhibitors (FIG. 14A and FIG. 14D) or basal differentiation (FIG. 14C).

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

Insulin resistance is necessary for the development of the metabolic syndrome and type 2 diabetes (T2D), and is a major risk factor for cardiovascular disease, which together represent a modern pandemic. While genome-wide association studies (GWAS) have identified a large number of genomic loci associated with T2D-related traits, most of these signals are associated with pancreatic β-cell function and insulin secretion rather than with insulin resistance². While a few insulin resistance genes have been identified, the underlying genetic architecture of insulin resistance remains unknown.

To fill this gap, the goal was to take advantage of a large library of induced pluripotent stem cells (iPSCs) derived from individuals across the spectrum of insulin sensitivity who have also undergone GWAS genotyping. these iPSC lines were fully characterized and demonstrated determinants of iPSC transcriptional variability. For instance, it was found that the highest across individual contribution to variability in the cohort was enriched for metabolic functions.

These results prompted us to more specifically analyze the gene expression patterns and networks associated with the insulin sensitivity status of the iPSC donors. For complex conditions like insulin resistance with polygenic susceptibility systems biology and network modeling, integrating multiscale-omics data like genetic and transcriptomic data, provide a useful context in which to interpret associations between genes and functional variation or disease states. Therefore, the reconstruction of molecular networks can lead to a more systematic and data driven characterization of pathways underlying disease, and consequently, a more comprehensive approach to identifying and prioritizing therapeutic targets. Recent advances in co-expression and causal/predictive network modeling allow us to take such an approach. The systems and methods herein link complex disease phenotypes from highly characterized subjects to concomitant molecular networks that can then be used to uncover coherent, functional molecular sub-networks and their key driver genes that ultimately determine the clinical phenotypes.

In summary, differential expression analyses were performed between insulin resistant (IR) and insulin sensitive (IS) iPSCs, built co-expression networks to systematically organize the data into coherent modules enriched for insulin sensitivity associated functions and implemented key driver analyses to identify genes that control and regulate critical aspects of the IR and IS networks. Finally, there was empirically validation of the constructed networks in iPSCs and the prioritized key drivers through insulin responsiveness associated functional assays in human adipocytes and skeletal muscle cells (SKMCs).

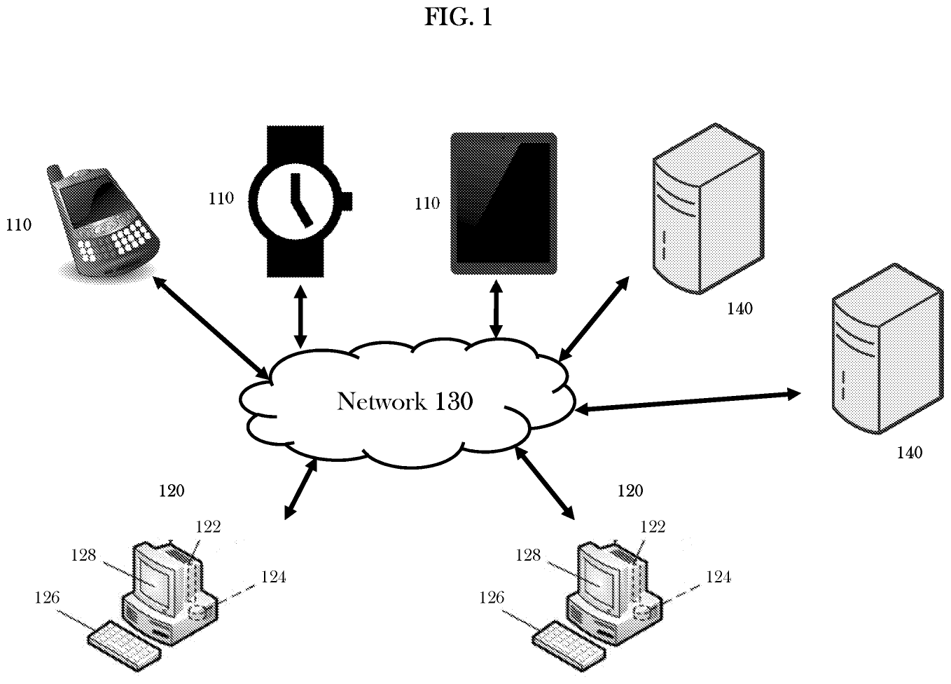

FIG. 1 is an exemplary embodiment of the hardware of the predictive network system. In the exemplary system 100, one or more peripheral devices 110 are connected to one or more computers 120 through a network 130. Examples of peripheral devices 110 include clocks, smartphones, tablets, wearable devices such as smartwatches, and any other networked devices that are known in the art. The network 130 may be a wide-area network, like the Internet, or a local area network, like an intranet. Because of the network 130, the physical location of the peripheral devices 110 and the computers 120 has no effect on the functionality of the hardware and software of the invention. Unless otherwise specified, it is contemplated that the peripheral devices 110 and the computers 120 may be in the same or in different physical locations. Communication between the hardware of the system may be accomplished in numerous known ways, for example using network connectivity components such as a modem or Ethernet adapter. The peripheral devices 110 and the computers 120 will both include or be attached to communication equipment. Communications are contemplated as occurring through industry-standard protocols such as HTTP.

Each computer 120 is comprised of a central processing unit 122, a storage medium 124, a user-input device 126, and a display 128. Examples of computers that may be used are: commercially available personal computers, open source computing devices (e.g. Raspberry Pi), commercially available servers, and commercially available portable device (e.g. smartphones, smartwatches, tablets). In one embodiment, each of the peripheral devices 110 and each of the computers 120 of the system may have the software related to the system installed on it. In such an embodiment, data may be stored locally on the networked computers 120 or alternately, on one or more remote servers 140 that are accessible to any of the networked computers 120 through a network 130. The remote servers 140 may store scientific or other databases that may be used by the disclosed invention. In alternate embodiments, the software runs as an application on the peripheral devices 110.

The systems and methods of the present invention build on existing work on pairwise causality to a full spectrum of causality inference in a multi-dimensional setting. In a network, it is particularly challenging to discern direct causality from correlations. The challenge of learning a causal network includes two sub-tasks: i) infer conditional independence among multiple variables; and ii) infer direct causality between two or more variables. In the case of pairwise causality inference, there have been excellent studies recruiting nonparametric models and an information geometric approach. In their approach, the causality is inferred by testing the independence between marginal distribution of cause and conditional distribution of response given cause. The independence is defined as orthogonality in information space based on relative entropy. This algorithm works particularly well if X and Y are deterministically related. However, if additive noise with a distribution closer to the reference, entropy-based IGCI with reference manifold, fails if the non-linearity of f is small compared to the width of the noise.

The present invention leverages an intuitive linear regression treatment encrypted in the frame of bottom-up Bayesian inference based on a set of constraints over the conditional probability distribution given hypothesis on causality direction and function. The constraints recapitulate an ensemble of possible linear regression fits. Based on that, it can be shown that linear calculations of the marginal probability of X and Y by constrained Bayesian belief propagation constitute an effective and intuitive approximation to the nonlinear information geometric measure to infer the causality direction from complex nonlinear function.

Bayesian Networks

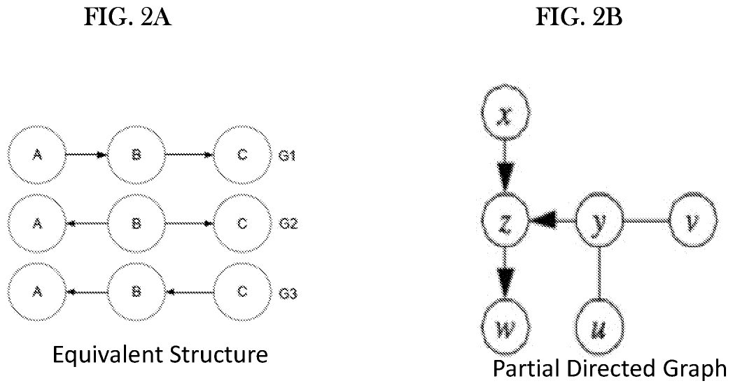

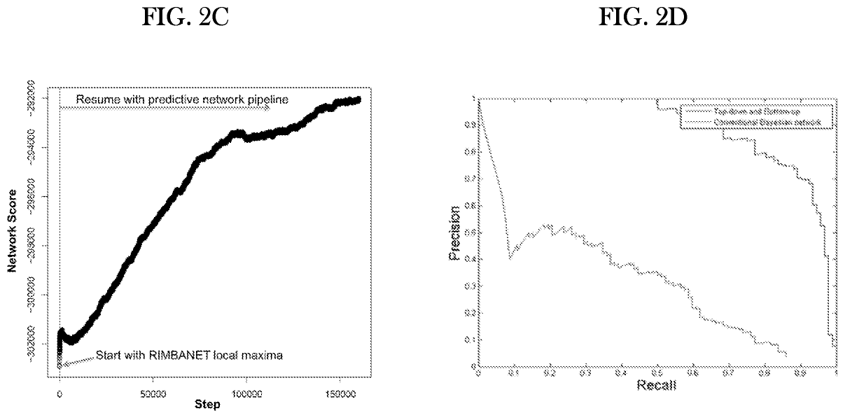

A Bayesian network represents a joint probability distribution over a set of random variables v={x₁, . . . , xₙ}, which are assumed to be categorical. It can be defined by a graph structure G and a vector of parameter θ, i.e. conditional probability distribution (CPD), i.e. m={G, θ}. The structure consists of a set of variables v={v₁, . . . , vₙ} and edges e={e₁, . . . , eₘ}, i.e. G={v, e}, where G is a directed acyclic graph (DAG); θ is a collection of conditional mass functions p(Xᵢ|πᵢ) where πᵢ denotes a set of parents of i-th node xᵢ in the graph respecting the relations of e. In a Bayesian network every variable is conditionally independent of its non-descendants given its parents, as shown in FIG. 2A.

The following notion is used by the present invention: Xᵢ represents i-th node or random variable in the network and xᵢₖ denotes the k-th state of Xᵢ whose state space contains rᵢ=|xᵢ| number of discrete values. πᵢⱼ represents the vector of j-th configuration on parents of Xᵢ. qᵢ=|πᵢ| denotes the number of total instantiations of the parent set πᵢ of Xᵢ. The model parameter θ={θᵢⱼₖ|∀ijk} is a vector of conditional probabilities where θᵢⱼₖ=p(xᵢₖ|πᵢⱼ), with i∈{1, . . . , n}, j∈{1, . . . , qᵢ}, k∈{1, . . . , rᵢ}. In the above, bold letters are used to denote vectors or sets, however, throughout this disclosure, bold and normal letters are used interchangeably wherever it is clear. The Bayesian network represents a joint probability distribution p(x)=p(x₁, . . . , xₙ)=Πᵢp(xᵢ|πᵢ), for every X in v.

Bayesian Network Structure Learning

A Bayesian network represents a joint probability distribution over a set of random variables v={x₁, . . . , xₙ}, which is assumed to be categorical. It can be defined by a graph structure G and a vector of parameter θ, i.e. conditional probability distribution (CPD), i.e. m={G, θ}. The structure consists of a set of variables v={v₁, . . . vₙ} and edges e={e₁, . . . , eₘ}, i.e. G={v, e}, where G is a directed acyclic graph (DAG); θ is a collection of conditional mass functions p(Xᵢ|πᵢ) where n denotes a set of parents of i-th node xᵢ in the graph respecting the relations of e. In a Bayesian network every variable is conditionally independent of its non-descendants given its parents. The Bayesian network represents a joint probability distribution p(x)=p(x₁, . . . , xₙ)=Πᵢp(xᵢ|πᵢ), for every X in v. A partial directed graph of this nature is shown in FIG. 2B.

Given observation data D, it is an objective of the present invention to reconstruct the structure of a Bayesian network. A score S (G) is assigned to the graph G to assess the fitness of a network G to the data set D. This score is given by the posterior probability as Eq. A1 below.

$$S ( G ) = \frac{P ( D \vert G ) P ( G )}{P ( D )}$$

where P (D|G) is the marginal data likelihood, P (G) is the prior probability of structure G and P (D) a normalizing constant. The marginal data likelihood can be calculated as Eq. A2 below.

P(D|G)=∫_{θ}P(D|θ,G,ξ)P(θ|G,ξ)dθ

where θ is the set of parameters and ξ indicates the prior background information. It is assumed that the data set D consists of N independent data samples d^{l}, then the data likelihood can be decomposed as Eq. A3 below.

$$P ( D \vert G ) = {\prod }_{l = 1}^{N} P ( {d}^{l} \vert θ , G , ξ )$$

Thus, Eq. A2 can be written as Eq. A4 below.

$$P ( D \vert G ) = {\prod }_{l = 1}^{N} {\int }_{θ} P ( {d}^{l} \vert θ , G , ξ ) P ( θ \vert G , ξ ) d θ$$

To solve the Eq. A4 in closed form, five assumptions are made.

Assumption 1 Multinomial Distribution: Let dᵢ^{l} and d_{π}_{ᵢ}^{l}, denote the variable Xᵢ and the parent set πᵢ in the l-th case of data set D, then,

P(d^{l}=k|d_{π}_{ᵢ}^{l}=j,θ,G,ξ)=θᵢⱼₖ,∈[0,1],∀Xᵢ,πᵢ

Assumption 2 Parameter Independence: Given network structure G, the parameters associated with each variable are independent from each other such that P(θ|G,ξ) decomposes into

$$P ( θ \vert G , ξ ) = {\prod }_{i = 1}^{n} P ( {θ}_{i} \vert G , ξ ) ∀ i = 1 , … , n$$

Since each instance of parents of a variable Xi are independent. P(θᵢ|G,ξ) decomposes into

$$P ( {θ}_{i} \vert G , ξ ) = {\prod }_{j = 1}^{{q}_{i}} P ( {θ}_{i j} \vert G , ξ ) ∀ i = 1 , … , n ; j = 1 , … , {q}_{i}$$

where qᵢ is the number of configurations the set of parents πᵢ can take.

Assumption 3 Parameter Modularity: Given two network structures G1 and G2, if Xi has the same parents in G1 and G2, then

P(θᵢⱼ|G₁,ξ)=P(θᵢⱼ|G₂,ξ)∀i=1, . . . ,n;j=1, . . .qᵢ

Assumption 4 Dirichlet Prior: Given a network structure G, P(θᵢⱼ|G₁,ξ), is a priori Dirichlet distributed, θᵢⱼ˜D(Nᵢⱼ₁, . . . , Nᵢⱼᵣ_{ᵢ}), exist exponents N′ᵢⱼₖ, which depend on

$$P ( {θ}_{i j} \vert G , ξ ) = \frac{Γ ( {Σ}_{k = 1}^{{r}_{i}} {N}_{i j k}^{′} )}{{Π}_{k = 1}^{{r}_{i}} Γ ( {N}_{i j k}^{′} )} {\prod }_{k} {θ}_{i j k}^{{N}_{ijk}^{′} - 1}$$

where Γ(*) denotes the Gamma function and rᵢ is the number of values of variable Xi. The hyper-parameters N′ᵢⱼₖ can be computed as:

N′ᵢⱼₖ=N′P(Xᵢ=k,πᵢ=j|ξ)

where N′ is the equivalent sample size and P(Xᵢ=k, πᵢ=j|ξ) is the prior joint probability distribution over the variable Xᵢ and its parents πᵢ.

Assumption 5 Complete Data: The data set is complete. That is, D contains no missing values or hidden variables. From the multinomial sample assumption and the assumption of complete data, P(D|θ,G) can be factorized into:

$$P ( D \vert θ , G , ξ ) = {\prod }_{l = 1}^{N} {\prod }_{i = 1}^{n} P ( {d}^{l} = k \vert {d}_{{π}_{i}}^{l} = j , θ , G , ξ ) = {\prod }_{i = 1}^{n} {\prod }_{j = 1}^{{q}_{i}} {\prod }_{k = 1}^{{r}_{i}} {θ}_{i j k}^{{N}_{ijk}}$$

where Nᵢⱼₖ equals to the number of the samples (Xᵢ=k, πᵢ=j) in D. Substituting Dirichlet prior and complete assumption into marginal data likelihood results in:

$$P ( D \vert G ) = {\int }_{θ} P ( D \vert θ , G , ξ ) P ( θ \vert G , ξ ) d θ = {\int }_{θ} {\prod }_{i = 1}^{n} {\prod }_{j = 1}^{{q}_{i}} {\prod }_{k = 1}^{{r}_{i}} {θ}_{ijk}^{{N}_{ijk}} \frac{Γ ( {\sum }_{k = 1}^{{r}_{i}} {N}_{ijk}^{′} )}{{\prod }_{k = 1}^{{r}_{i}} Γ ( {N}_{ijk}^{′} )} {\prod }_{k} {θ}_{ijk}^{{N}_{ijk}^{′} - 1} = {\prod }_{i = 1}^{n} {\prod }_{j = 1}^{{q}_{i}} \frac{Γ ( {\sum }_{k = 1}^{{r}_{i}} {N}_{ijk}^{′} )}{{\prod }_{k = 1}^{{r}_{i}} Γ ( {N}_{ijk}^{′} )} {\int }_{θ} {\prod }_{k = 1}^{{r}_{i}} {θ}_{ijk}^{{N}_{ijk} + {N}_{ijk}^{′} - 1} d θ$$

The posterior of each parameter remains in the conjugate family since the Dirichlet distribution is conjugate for this domain. The integral equals:

$${\int }_{θ} {\prod }_{k = 1}^{{r}_{i}} {θ}_{i j k}^{{N}_{ijk} + {N}_{ijk}^{′} - 1} d θ = \frac{{Π}_{k = 1}^{{r}_{i}} Γ ( {N}_{ijk} + {N}_{i j k}^{′} )}{Γ ( {N}_{i j} + {N}_{i j}^{′} )}$$

where Nᵢⱼ=Σₖ Nᵢⱼₖ and N′ᵢⱼ=Σₖ N′ᵢⱼₖ Thus, the marginal data likelihood reads

$$P ( D \vert G ) = {\prod }_{i = 1}^{n} {\prod }_{j = 1}^{{q}_{i}} \frac{Γ ( {N}_{ij}^{′} )}{Γ ( {N}_{ij} + {N}_{ij}^{′} )} {\prod }_{k = 1}^{{r}_{i}} \frac{Γ ( {N}_{ijk} + {N}_{ijk}^{′} )}{Γ ( {N}_{ijk}^{′} )} \qquad ( 0 )$$

Bottom-Up Causality Inference

In the case of a nonlinear relationship between X and Y, the nonlinearity in the function defining the relationship between cause X and effect Y, i.e. Y=f(X), can also be considered for causal inference in the presence of additive noise. The nonlinearity provides information on the underlying causal model and thus allows more aspects of the true causal mechanism to be identified. Previously, a method for functional causal modeling was proposed where the inherent probabilistic inference capability of the Bayesian network framework was integrated to generate predictions of hypothesized child (response) nodes using the observed data of the hypothesized parent (causal) nodes. By defining a distance metric in probability space that assesses how well the predicted distribution of child nodes matches the distribution of their observed values, the different causality configurations within an equivalence class can be evaluated to determine the one best supported by the data. One key advantage of this modeling approach is that it enables the propagation of the effects of a parent node to child nodes that can be greater than a path length of one from the parent, thereby making it possible to infer causality in a chain of nodes or in a more complex network structures.

To describe the approach of the present invention, the process begins by reformatting the general posterior probability corresponding to any given graphical structure as defined in conventional Bayesian network approaches. Let X represent the vector of variables represented as nodes in the network; E is the evidence; D denotes the observed data; G is the graphical network structure to infer; and θ is a vector of model parameters. From this the posterior probability may be written as P(G|D)=P(D|G)P(G)/P(D), where the marginal probability P(D|G) can be expressed as an integral over the parameters, given a particular graphical structure G: P(D|G)=∫_{θ}P(D|G,θ)P(θ|G)dθ. Unlike the traditional Bayesian Dirichlet score, D in this case contains continuous values, and thus, the likelihood of the data is not derived from a multi-nominal distribution, but rather a continuous density function whose form is estimated using a kernel density estimation procedure. In addition, the parameter prior P (Γ|G) does not follow a Dirichlet distribution but rather is either described by a set of non-parametric constraints in parameter space or is sampled from a uniform distribution defined in its range (the approach used herein). Given this, the above integral has no analytical solution. The data likelihood is then optimized by estimating θ using maximum-a-posteriori (MAP) estimation:

P(D|G)=∫_{θ}P(D|G,θ)P(θ|G)dθ|P(D|G,\hat{θ}) (1)

where \hat{θ}=argmax {P(D|G,θ)P(θ|G)}. P(D|G, θ) equals to the data likelihood and P(θ|G) denotes the prior distribution over parameters. A Monte Carlo sampling procedure is used to efficiently sample θ from P(θ|G), evaluating the likelihood for each parameter sample.

Deriving Bottom-Up Data Likelihood Score

To calculate and optimize the data likelihood P(D|G, θ), belief propagation as a subroutine is incorporated in the bottom-up causal inference procedure to predict the marginal probabilities of all response variables given the observed data for the predictor variables for a given causal structure/configuration (G). In this instance, the marginal probability of X given G and the parameter θ is calculated via belief propagation. The following disclosure explains how to exemplarily use the Bayesian belief inference P(X|E, G, θ) to calculate the data likelihood P(D|G, θ) in Eq. 1. Where E and D represent the observed data on the parent and child nodes, respectively. First, to avoid confusion, X_{b} is used herein to describe the binary variable in the probability space mapped from the continuous variable X∈R. Second, the original observation data is rescaled so that it falls in the interval, as explained further below. Third, a hidden variable H is introduced to fully specify the data likelihood as:

P(D|G,θ)=∫_{H}P(D|G,H,θ)P(H|G,θ)dH (2)

Given G and θ, the soft evidence enters P(X_{b}|E, G, θ) as the observed, rescaled data D, which effectively “clamps” (or fixes) the marginal probability of the parent nodes; the marginal probability of the child nodes is predicted via belief propagation in the Bayesian network. These marginal probabilities are then used to define the hidden data H, which are used to construct the marginal data likelihood in Eq. 2. In probability space, the belief inference is deterministic, i.e. given a causal structure G, a specific set of parameters θ, and evidence E, P(X_{b}|E, G, θ) is uniquely determined. In Eq. 2, when H=P(X_{b}|E, G, θ), P(H|G,θ)=1 and 0 otherwise, and as a result the data marginal likelihood in Eq. 2 can be re-written as

P(D|G,θ)=P(D|H(G,θ))=P(D|P(X_{b}|E,G,θ)) (3)

The inner probability describes the marginal belief of the binary variable X_{b} in probability space to which the original continuous variable X has been mapped. This belief probability is a linear function between the child and parent marginal probabilities, multiplied by the conditional probabilities determined by sampling over the uniform distribution the parameters θ are assumed to follow:

P(X_{b}^{chd}=k)=Σᵢ[P(X_{b}^{chd}=k|X_{b}^{π}=Cᵢ)P(X_{b}^{π}=Cᵢ)] (4)

with X_{b}^{π} representing X_{b} for the parent node, X_{b}^{chd} for the child node, and Cᵢ represents the i-th conjuration of the parent nodes. For example, given the hypothesis “gene A regulates gene B”, the marginal belief is calculated as:

P(B)=P(B|A)P(A)+P(B|Ā)(1−P(A))=[P(B|A)−P(B|Ā)]P(A)+P(B|Ā) (5)

In this instance, the conditional probability distribution θ={P(B|A), P(B|Ā)} is the parameter sampled from the uniform distribution on [0,1]. Note that this belief propagation can be considered as a linear regression of the form P(B)=βP(A)+C, where β=(P(B|A)−P(B|Ā)) and C=P(B|Ā). Given probability measures are constrained to be between 0 and 1, β∈[−1, +1] and C∈[0,1]. These constraints, which follow naturally from probability theory, give rise to the asymmetric basis of the approach disclosed herein for causal inference. It is further notable that in the approach is implicitly assumed that binary variables in probability space, where the belief probability of a binary variable is defined as the level of belief on that variable observed in its maximal state (i.e., P(X_{b}=1)) or minimal state (i.e., P(X_{b}=0)). When X is equal to its minimum (or maximum) value in the real valued space D, in probability space X_{b} is observed in its minimum (or maximum) state, and therefore, this observed sample will correspond to P(X_{b}=0)=1 (or P(X_{b}=0)=0) in probability space. As the value of X varies between its minimum and maximum values, the belief probability of the binary mapping of this variable will vary between [0,1]. The Bayesian interpretation of the belief probability allows for a comparison of the inferred belief probability to the real-valued observed data by implicitly assuming that the original data D and the marginal probabilities of H are positively correlated, though the precise kinetics of this correlation is unknown. To make such a comparison, rescale D∈R to D∈[0,1].

Since the exact function mapping between real values and their belief probabilities is unknown, a non-parametric metric, i.e. Kullback-Liebler (KL) divergence is employed to compare the distribution of real observations to the distribution of predicted marginal probabilities. If the predicted and observed distribution of the child nodes match well, it can be concluded that the predictions based on G and θ well reflect the observed data D, which results in a smaller value of the KL-divergence. To force the KL divergence to behave as a true probability measure, symmetry and normalization modifications are made to this function defined on D and H such that k(D;H)=1−exp[−(KL(D∥H)+KL(H∥D))/2]. The data likelihood function in Eq. 3 can be defined by any normalized monotonic decreasing function on the kernel. For model selection, where S=−log(K(D, H)) to represent the posterior score of the model, which is negatively correlated with the kernel value. To optimize the model, this score is maximized, which is equivalent to minimizing the KL-divergence.

The calculation of the KL-divergence involves comparing the real-valued observed data D to the inferred belief probabilities H given a particular causal hypothesis G and θ. The original interaction between parent(s) and child X_{chd} nodes in D can be described by an arbitrary function X_{chd}=μ(X_{π}) plus some observation noise. Depending on the nature of causal relationships to be modeled, μ( ) can take various forms, including linear, non-linear, monotonic, non-monotonic, concave, convex, step and periodic functions. In the biology domain, a direct causal interaction between two proteins or between a protein and DNA molecule often take the form of a hill function, a step function, or a more general non-monotonic, nonlinear function.

One way to derive the belief inference given in Eq. 4 is to represent the relationship between the parent and child nodes as a cubic spline, which can well approximate general nonlinear relationships. However, a more straightforward alternative to splines would be regressing the marginal belief of X_{chd} onto X_{π}, assuming a linear relationship. If the range of the parent nodes is subdivided into L segments based upon the behavior a causal relationship between two nodes is expected, and linear regressions can be carried out in each segment. In the case |π|=1, the problem is to regress onto a single variable whose range has been divided into L segments, whereas when |π|>1 is regressed onto multiple variables divided into a |π|-dimensional grid comprised of L^{|π|} components. Here, the focus is only on inferring causality between equivalent class structures in which |π|=1, but the approach easily extends to the more general case.

To derive a procedure for fitting the belief inference equation to the data in a piecewise fashion, some terms must first be defined. Let X denote a vector of predictor(s)/parent node(s) (in this pair-wise causality setting X represents just a single variable for any given Markov equivalent structure being considered) and let Y represent the response variables. Let D∈Rⁿ represent the original noisy observed data and D₀^{l}∈[0,1]ⁿ denote the rescaled observed data in the l-th segment/grid element, which is comprised of the observed values over the parent and child nodes in the given segment/grid element, i.e. D₀^{l}={D_{X}^{l}, D_{Y}^{l}}. Similarly, let the predicted data in the l-th segment be H={H_{X}^{l}, H_{Y}^{l}}. Given this, one can pre-define a total of K bins that are evenly distributed in [0,1], I^{k}=[k/k, (k+1)/K], k=[0, K−1], and then for each bin the number of occurrences of the inferred marginal probability of Y falling in each of the 1 segments is counted, i.e. H_{Y}^{l} falls in the k-th bin I^{k}∈[0,1]. The number of occurrences for the k-th bin and the l-th segment/grid element is denoted by Mₖ^{l}. The counterpart of this number in D_{Y}^{l}, with respect to the observed data, is denoted by Nₖ^{l}. The frequency for the predicted data is then calculated as pₖ^{l}=Mₖ^{l}/ΣₖMₖ^{l} for H_{Y}^{l} and similarly for the observed data, D_{Y}^{l}, qₖ^{l}=Nₖ^{l}/Σₖ Nₖ^{l}. These counts and frequencies are used to compute the KL-divergence kernel, maximize the likelihood score, and identify the maximum LR model \hat{θ} in Eq. 1 per segment as described below. To maximize the data likelihood function P(D|G, \hat{θ}) in Eq. 1 in each segment, the parameter that minimizes the KL-divergence for the current causal hypothesis G is identified, which is defined as the symmetrized KL-divergence between the predicted belief and the rescaled observed data for every segment:

$${\hat{θ}}^{l} = {arg max}_{θ} \{ P ( {D}_{0}^{l} \vert G , θ ) P ( θ \vert G ) \} ∝ {arg max}_{θ} \{ P ( {D}_{Y}^{l} \vert {H}_{Y}^{l} , G , θ ) P ( θ \vert G ) \} ∝ {arg max}_{θ} \{ P ( {D}_{Y}^{l} \vert P ( Y \vert E = {D}_{X}^{l} , G , θ ) ) \} ∝ {arg max}_{θ} \{ P ( k ( {D}_{Y}^{l} , {H}_{Y}^{l} ( G , θ ) ) ) \} ∝ {arg max}_{θ} \{ - log ( k ( {D}_{Y}^{l} , {H}_{Y}^{l} ( G , θ ) ) ) \} ∝ {arg min}_{θ} \{ {\sum }_{k = 0}^{K} {p}_{k}^{l} ln ( \frac{{p}_{k}^{l}}{{q}_{k}^{l}} ) + {\sum }_{k = 0}^{K} {q}_{k}^{l} ln ( \frac{{q}_{k}^{l}}{{p}_{k}^{l}} ) \}$$

where P (θ|G)=1/M for θ sampled uniformly in [0,1]. The statistical counts of the predicted probability, pₖ^{l} for l-th segment in k-th bin, is a function of G and θ. The optimal statistical count of the fitted model for the l-th segment and k-th bin is \hat{M}ₖ^{l}. The overall fitted linear regression model (G, θ^{l}|l=1, . . . , L) with the counts across all segments in k-th bin of [0,1] is obtained by summing \hat{M}ₖ^{l} over the total L segments, i.e. \hat{M}ₖ=Σₜ₌₁^{L} \hat{M}ₖ^{l}. According to Eq. 1 and Eq. 3, the final optimized estimation of the data likelihood is then equal to

$$P ( D \vert G , \hat{θ} ) ∝ - log ( 1 - exp ( - \frac{1}{2} [ {\sum }_{k = 0}^{K} {\hat{p}}_{κ} ln ( \frac{{\hat{\hat{p}}}_{k}}{{q}_{k}} ) + {\sum }_{k = 0}^{K} {q}_{k} ln ( \frac{{\hat{q}}_{k}}{{\hat{p}}_{k}} ) ] ) \qquad ( 7 )$$

For simplicity, in the experiment section below, to obtain the L segments in [0,1], the range of each parent node can be divided evenly into L segments.

Top-Down & Bottom-Up Predictive Network Learning Algorithm

At a very high level, the predictive network learning algorithm used by the present invention can be generalized as (1) checking if the after-move structure is equivalent to the before-move; and (2) if that check returns as true, calculate the Bottom-up score for the after-and-before move structure and use that to determine the causality.

Top-Down & Bottom-Up Scoring

The top-down & bottom-up predictive network algorithm integrates conventional top-down Bayesian scoring, discussed and the novel bottom-up prediction score to infer causal network topology. In particular, the bottom-up score will be used to infer causality among equivalent structures while top-down score is used to infer the conditional independence.

To clarify the algorithm, the following notation is defined: Assume the original observation (continuous) data to be D₀, and its rescaled form to [0,1] is denoted by D_{c} (‘c’ stands for continuous) for bottom-up prediction score. Dₒ is also discretized into categorical values denoted by D_{d} (‘d’ stands for discrete) for top-down Bayesian score. The top-down & bottom-up score is defined by the posterior probability of the structure G given the rescaled continuous data D_{c} and the discretized data D_{d} as

$$P ( G \vert {D}_{c} , {D}_{d} ) = \frac{P ( {D}_{c} , {D}_{d} \vert G ) P ( G )}{P ( {D}_{c} , {D}_{d} )} ∝ P ( {D}_{c} , {D}_{d} \vert G ) \qquad ( 8 )$$

The marginalized data likelihood can be written as

P(D_{c},D_{d}|G)=∫_{θ}P(D_{c},D_{d}|G,θ)P(θ|G)dθ (9)

D_{d} is discretized data D₀, the assumption of multinomial distribution on D_{d} is taken as Bayesian score (as described in section 3.1.1): D_{d}={d_{i,s}^{d}, d_{π}_{ᵢ}_{,s}^{d}}, where i=1 . . . N and s=1 . . . S. d_{i,s}^{d} denote the discrete value of variable Xᵢ and d_{π}_{ᵢ}_{,s}^{d} represents the discrete value of parent set πᵢ in the s-th case of data set D_{d}. Similarly, D_{c} denotes the matrix of rescaled data D_{C}={d_{i,s}^{c}, d_{π}_{ᵢ}_{,s}^{c}}. The joint data likelihood in Eq. 9 can be written as:

P(D_{c},D_{d}|G,θ)=P(d_{i,s}^{d},d_{π}_{ᵢ}_{,s}^{d},d_{i,s}^{c},d_{π}_{ᵢ}^{c}|G,θ) (10)

As the discretized and rescaled data are dependent, a hidden variable I is introduced to make the joint data likelihood decomposable.

P(d_{i,s}^{d},d_{π}_{ᵢ}_{,s}^{d},d_{i,s}^{c},d_{π}_{ᵢ}^{c}|G,θ)=∫_{I}P(d_{i,s}^{d},d_{π}_{ᵢ}_{,s}^{d},d_{i,s}^{c},d_{π}_{ᵢ}^{c}|I,G,θ)P(I|G,θ)dI

where I represents a binary indicator dependent only on current network structure (G).

$$P ( {d}_{i , s}^{d} , {d}_{{π}_{{ }^{i}} , s}^{d} , {d}_{i , s}^{c} , {d}_{{π}_{i}}^{c} \vert G , θ ) = P ( {d}_{i , s}^{d} , {d}_{{π}_{{ }^{i}} , s}^{d} , {d}_{i , s}^{c} , {d}_{{π}_{i}}^{c} \vert I = 1 , G , θ ) P ( I = 1 \vert G , θ ) + P ( {d}_{i , s}^{d} , {d}_{{π}_{i} , s}^{d} , {d}_{i , s}^{c} , {d}_{{π}_{i}}^{c} \vert I = 0 , G , θ ) P ( I = 0 \vert G , θ )$$

If current network structure G belongs to any equivalent structure class, then I=1, i.e. P(I=1|G,θ)=P(I=1|G)=1 and P(I=0|G,θ)=P=(I=0|G)=0; If current network structure G doesn't belong to any equivalent structure class, then I=0, i.e. P(I=1|G,θ)=P(I=1|G)=0 and P=(I=0|G,θ)=P(I=0|G)=1. We further define that:

P(d_{i,s}^{d},d_{π}_{ᵢ}_{,s}^{d},d_{i,s}^{c},d_{π}_{ᵢ}^{c}|I=1,G,θ)=P(d_{i,s}^{c},d_{π}_{ᵢ}^{c}|G,θ)

P(d_{i,s}^{d},d_{π}_{ᵢ}_{,s}^{d},d_{i,s}^{c},d_{π}_{ᵢ}^{c}|I=0,G,θ)=P(d_{i,s}^{d},d_{π}_{ᵢ}^{d}|G,θ)

therefore, the integrated data likelihood can be written as:

P(d_{i,s}^{d},d_{π}_{ᵢ}_{,s}^{d},d_{i,s}^{c},d_{π}_{ᵢ}^{c}|G,θ)=P(d_{i,s}^{d},d_{π}_{ᵢ}^{d}|G,θ)P(I=0|G,θ+P(d_{i,s}^{c},d_{π}_{ᵢ}^{c}|G,θ)P(I=1|G) (11)

where P(d_{i,s}^{d}, d_{π}_{ᵢ}_{,s}^{d}|G,θ)=P(d_{i,s}^{d}=k|d_{π}_{ᵢ}_{,s}^{d}=j,θ,G)=θᵢⱼₖ, which is equivalent to the data-likelihood in Bayesian network (section 3.1.1). P(d_{i,s}^{c}, d_{π}_{ᵢ}^{c}|G,θ)=P(d_{i,s}^{c}, d_{π}_{ᵢ}_{,s}^{c}|H(G, θ)=P(d_{i,s}^{c}, d_{π}_{ᵢ}^{c}|P(Xᵢ|E=d_{π}_{ᵢ}^{c},G,θ)) (see Eq. 3 in the above description) in the second term equals to the bottom-up prediction score.

Therefore, the overall top-down & bottom-up score in Eq. 9 can be written as:

$$P ( {D}_{c} , {D}_{d} \vert G ) = {\int }_{θ} P ( {D}_{c} , {D}_{d} \vert G , θ ) P ( θ \vert G ) d θ = {\int }_{θ} {\int }_{I} P ( {d}_{i , s}^{d} , {d}_{{π}_{i} , s ,}^{d} {d}_{i , s}^{c} , {d}_{{π}_{i}}^{c} \vert I , G , θ ) P ( I \vert G ) P ( θ \vert G ) dId θ = {\int }_{θ} [ P ( {d}_{i , s}^{d} , {d}_{{π}_{i} , s}^{d} \vert G , θ ) P ( I = 0 \vert G ) + P ( {d}_{i , s}^{c} , {d}_{{π}_{i}}^{c} \vert G , θ ) P ( I = 1 \vert G ) ] P ( θ \vert G ) d θ = {\int }_{θ} P ( {d}_{i , s}^{d} , {d}_{{π}_{i} , s}^{d} \vert G , θ ) P ( θ \vert G ) d θ * P ( I = 0 \vert G ) + {\int }_{θ} ( {d}_{i , s}^{c} , {d}_{{π}_{i}}^{c} \vert G , θ ) P ( θ \vert G ) d θ * P ( I = 1 \vert G ) \qquad ( 12 )$$

The first term in Eq. 12 represents the (top-down) Bayesian Dirichlet score in Bayesian network and the second term in Eq. 12 denotes the (bottom-up) prediction score, as developed above. Final top-down & bottom-up score can be written as:

$$P ( G \vert {D}_{c} , {D}_{d} ) ∝ P ( {D}_{c} , {D}_{d} \vert G ) ∝ [ {\prod }_{i = 1}^{n} {\prod }_{j = 1}^{{q}_{i}} \frac{Γ ( {N}_{ij}^{′} )}{Γ ( {N}_{ij} + {N}_{ij}^{′} )} {\prod }_{k = 1}^{{r}_{i}} \frac{Γ ( {N}_{i j k} + {N}_{i j k}^{′} )}{Γ ( {N}_{i j k}^{′} )} ] * P ( I = 0 \vert G ) + [ - log ( 1 - exp ( - \frac{1}{2} [ {\sum }_{k = 0}^{K} {\hat{p}}_{κ} ln ( \frac{{\hat{\hat{p}}}_{k}}{{q}_{k}} ) + {\sum }_{k = 0}^{K} {q}_{k} ln ( \frac{{\hat{q}}_{k}}{{\hat{p}}_{k}} ) ] ) ] * P ( I = 1 \vert G ) \qquad ( 13 )$$

where the variables in the first term is defined as in Eq. 0 and variables in the second term is defined as in Eq. 7.

The final posterior in the top-down & bottom-up (“TDBU”) method is a combination of the original top-down & bottom-up score. As in Bayesian network, optimizing this score function (Eq. 13) is an NP-hard problem due to the size of the possible structure space. However, any heuristic search method, such as Monte Carlo Markov Chain (“MCMC”) or Hill Climbing etc., can be applied to optimize this posterior function. FIGS. 2C and 2D exemplarily show network scoring, and the improvement over standard Bayesian techniques in the precision and recall using the algorithms of the present invention, respectively.

### EXAMPLE 1

### Predictive Network Analysis Identifies HSPA2 as a Novel Alzheimer's Disease Target

It is believed that changes in gene and protein expression are crucial to the development of late onset Alzheimer's disease (LOAD). Herein, proteins are examined and incorporated into networks in two separate series, and the outputs are evaluated in two different cell lines. The pipeline included the following steps: (1) Predicting expression quantitative trait loci (eQTLs); (2) Determining differential expression; (3) Analyzing networks of transcript and peptide relationships; and (4) Validating effects in two separate cell lines. We performed all the analysis in two separate brain series to validate effects. The two series included 345 samples in the first set (177 controls, 168 cases; age range 65-105; 58% female; KRONOSII cohort) and 409 samples in the replicate set (153 controls, 141 cases, 115 MCI; age range 66-107; 63% female; RUSH cohort). The top target is Heat Shock Protein Family A Member 2 (HSPA2), which was identified as a key driver in the two datasets. HSPA2 was validated in two cell lines, with overexpression driving further elevation of ABeta40 and ABeta42 levels in APP mutant cells as well as significant elevation of Tau and phospho-Tau in a modified neuroglioma line. This work further demonstrates that studying changes in gene and protein expression is crucial to understanding late onset disease and further nominates HSPA2 as a specific key regulator of LOAD processes.

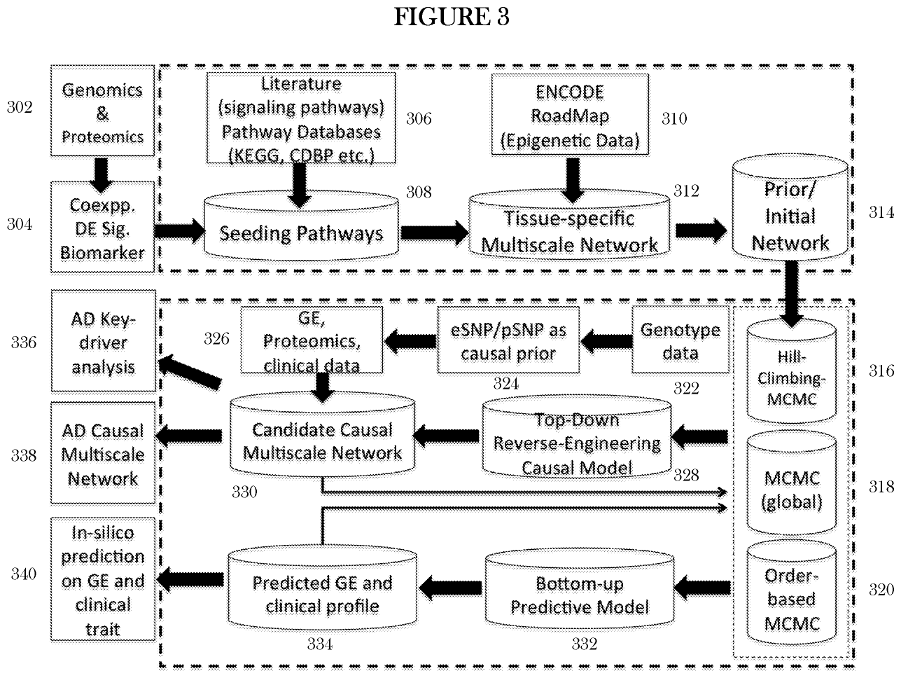

FIG. 3 discloses an exemplary logic flow in accordance with an embodiment of the invention, applying the above-mentioned equations and processes to biochemical drug target identification for Alzheimer's Disease (AD). Operation of the software of the present invention takes place at any of the computers 120 or peripheral devices 110 of the system. At step 302, the algorithm commences by collecting genetics, genomics and proteomics data from external databases. At step 304, the software collects coexpression and/or Differentially Expressed (DE) gene signature, and/or other biomarkers. At step 306, the software queries external literature and pathway databases, collecting additional data (optional) for the predictive network. At step 308, the software uses all of the data collected to form seeding pathways, in accordance with the foregoing disclosure. The epigenetic data, such as but not limited to ENCODE, RoadMap, is incorporated at step 310, creating a tissue-specific multiscale network, shown as step 312.

At step 314, a prior/initial network is created from the tissue-specific multiscale network. Overall the prior/initial network is optional and not needed in order to the following steps to work. In case no initial network is available or constructed before, the invented algorithm will directly incorporate genomics data, proteomics data at step 302 with the genotype data at step 322. If initial network is constructed, that prior/initial network is passed through any existing heuristic search method such as but not limited to hill-climbing, MCMC at step 316, a global MCMC at step 318, and an order-based MCMC at step 320. Interactively, at each step of heuristic search, the invented algorithm calculated the integrated top-down score at step 328 and bottom-up predictive score shown as step 332. Genotype data 322, eSNP/pSNP causal prior data 324, and GE/proteomics clinical data 326 is incorporated into the top-down reverse-engineering score at 328 to arrive at a candidate causal multiscale network 330. Similarly, the bottom-up predictive score 332 is used to calculate a predicted GE and clinical profile 334. Both the top-down score (328&330) and predictive score (332&334) are periodically passed to the hill-climbing, MCMC at step 316, the global MCMC at step 318, and the order-based MCMC at step 320 to update them as the progress of structure learning by the software. The final output of this learning process is the integrated top-down & bottom-up predictive network model by optimizing the combined top-down & bottom-up score. The final predictive network is used to determine the AD key-driver analysis 336 and the AD causal multiscale network 338, while the predicted GE and clinical profile 334 is used to calculate in-silico prediction on GE and clinical trait 340. The results are described in greater detail below.

SAMPLES: KRONOSII is a subset of data already presented (Corneveaux et al., 2010) and contains samples from Alzheimer's Disease Research Center funded US brain banks as well as 6 European and British brain banks. KRONOSII is a convenience cohort with low secondary pathology (i.e. Lewy body disease) and high pathology load in the LOAD affected samples and low pathology load for controls. The second set (RUSH) includes subjects from two large, prospectively followed cohorts maintained by investigators at Rush University Medical Center in Chicago, Ill.: The Religious Orders Study and the Memory and Aging Project. The RUSH set is an epidemiologically based cohort with a greater mix of pathologies and pathological staging. There are 168 LOAD affected samples and 177 unaffected samples with all datasets collected for the KRONOSII cohort. There are 141 LOAD affected samples and 153 unaffected samples with all datasets collected for the RUSHII cohort. The average age for the KRONOSII cohort is 81, with 59% female subjects. The average age of the RUSH cohort is 88 and 63% of the subjects are female. Tissue sections were taken from frontal (82% of the sample) and temporal (18% of the sample) cortical regions.

DATA COLLECTION: Genomic DNA samples were analyzed on the Genome-Wide Human SNP 6.0 Array (Affymetrix, Inc. Santa Clara, Calif.) according to the manufacturer's protocols. Birdsuite (Korn et al., 2008) was used to call SNP genotypes from CEL files. The DNA quality control pipeline was similar to that described in Anderson et al (Anderson et al., 2010). cRNA was hybridized to Illumina HumanRefseq-HT-12 v2 Expression BeadChip (Illumina, San Diego, Calif.). Expression profiles were extracted, background was subtracted and missing bead types imputed using the BeadStudio software. Normalization for the RNA profiles was performed using lumi (Du et al., 2008) and limma (Ritchie et al., 2015). MS/MS analysis was performed using an Exactive Orbitrap mass spectrometer (Thermo Scientific, San Jose, Calif.) outfitted with a custom electrospray ionization (ESI) interface. Identification and quantification of peptides was performed using the accurate mass and time (AMT) tag approach (Zimmer et al., 2006). Decon2LS was used for peak-picking and for determining isotopic distributions and charge states (Jaitly et al., 2009). Deisotoped spectral information was loaded into VIPER to find and match features to the peptide identifications in the AMT tag database (Monroe et al., 2007). The area under the curve from extracted ion chromatograms was used as the measure of peptide abundance.

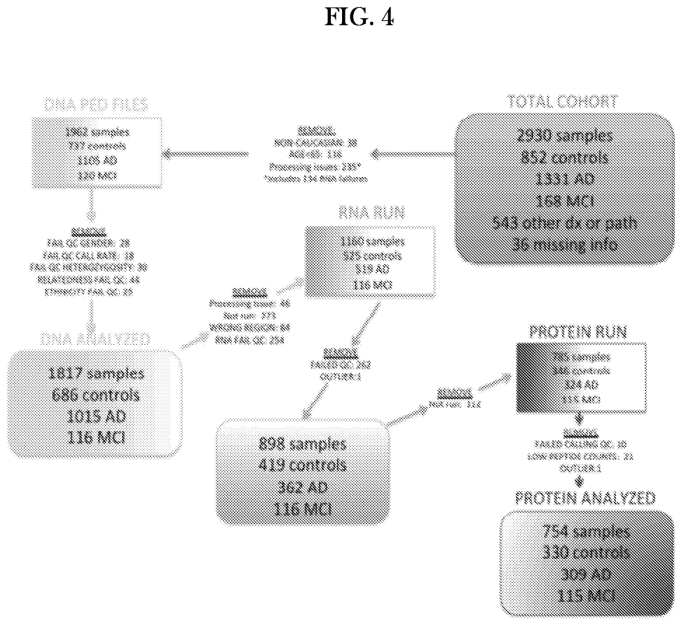

DATA ANALYSIS: The data analysis pipeline is shown in FIG. 4. This was a multi-pass selection procedure to both uncover LOAD risk targets and place them in the context of upstream regulation (allelic information) and downstream outputs (transcripts and peptides). The goal was to identify a minimal set of high-confidence targets for validation. The pipeline was performed in KRONOSII and RUSH separately after normalization to ensure independent replication.

Differential Expression (DE): DE analysis was performed using limma (Ritchie et al., 2015) comparing LOAD and pathologically confirmed controls. Each dataset (KRONOSII, RUSH) was run independently. Multiple testing adjustment was performed using Benjamini-Hochberg correction (5% FDR). Results were used to define seeding sets for downstream analysis.

Expression Quantitative Trait loci (eQTL): MatrixeQTL (Shabalin, 2012) was used to predict allele-transcript relationships. Each dataset (KRONOSII, RUSH) was run independently. Multiple testing adjustment was performed using Benjamini-Hochberg correction (5% FUR). Results were used to define seeding sets for downstream analysis.

Network Analysis: Network analyses was carried out, taking place as input genomic, transcriptomic and proteomic profiles from the two datasets (KRONOSII and RUSH), in addition to external data derived from the literature, pathway databases (mSIGDB, GO), and the Roadmap initiatives (Roadmap Epigenomics et al., 2015). The goal was to produce an output list of the main biological processes that are dysregulated in LOAD, as well as a small list of the top KDs impacting LOAD-associated processes. KRONOSII and RUSH were treated as independent datasets and the effects were compared across sets to determine replicated targets. The pipeline included the following procedures (FIG. 4, steps 3 to 6, dark orange squares): 1. Constructing COENs to identify sets of co-regulated genes associated with LOAD pathology (Step 3) and determining pathways enriched in each network module (Step 4b); 2. Determining seeding gene sets associated with LOAD pathology (Step 4a, Module Selection (MS) and Module Enrichment (ME)); 3. Building multi-scale CPNs (Step 5); and 4. Determining the KDs that modulate states of the CPN subnetworks (Step 6).

Causal Predictive Networks (CPN): While COENs allow for descriptive characterizations of gene-protein relationships, causal relationships prediction is necessary for ordering of the network data into a hierarchy of relationships that in turn enables KD analyses. While COENs reflect only associative relationships, BNs infer directed edges that represent the direction of information flow. BN analysis can capture nonlinear and combinatorial interactions. One limitation to standard BN analysis is that sometimes substructures within a BN are contradictory, which results in many directed edges having low confidence. To address this inherent limitation, a novel CPN approach was developed, integrating a top-down BN with bottom-up causal inference that considers known causal relationships that breaks the symmetry among contradictory causal structures and thus leads to higher confidence in edge directions.

The complexity of network building is a function of the number of nodes considered and sample size. All peptides in the network constructions were used; however, given the large number of probes used to query gene expression levels, the number of probes to use in the CPN reconstruction were reduced without losing important LOAD gene and pathway information. Gene-only COENs were built and identified those modules enriched for DE genes, and then restricted CPN construction to this subset of coherent, LOAD-focused gene sets.

The search was focused on the identification of key drivers of network states associated with LOAD, and thus used only LOAD datasets. The seeding gene sets for both the KRONOSII and RUSH LOAD datasets included modules enriched for DE transcript targets; therefore, pathways of relevance for LOAD pathology were selected. These sets were expanded to include more than just DE transcripts by including priors from a literature based brain specific network. All peptides were used in the network models given the modest number of peptides measured and their potential to link network components to pathways containing DE transcripts. Transcript data was reduced to the most crucial targets (Module Selection) and then expanded by including additional targets from the same pathways in curated databases (Module Enrichment). To ensure robust replication, KRONOSII and RUSH were pipelined as separate sets.

Key Driver Analysis (KDA): After the CPN analysis was performed, the resulting predictive network models were examined using a KDA algorithm. KDs are targets that have a significant impact on the regulatory states of other targets. KDs were predicted separately for KRONOSII and RUSH and overlaps determined. The LOAD-associated subnetworks to which KDA was applied were generated by projecting three different datasets onto the networks. First, all DE transcripts that overlapped between KRONOSII and RUSH were projected against both the transcript-only and transcript-peptide causal networks. Second, the DE products broken down in subnetworks corresponding to LOAD and control modules from the COENs were projected to determine how control effects were acting in LOAD predicted structures as well as enrich the DE dataset. Finally, the entire peptide set was projected onto the transcript-peptide causal network.

DATA VALIDATION: Of the eight targets, one target (ST18) was not followed due to construct size and cost. The other seven constructs were tested in the HEK293 and H4 lines. One construct (CP110) gave low transduction efficiencies due to construct size and was not followed (data not shown). Of the other targets, three (HSPA2, GNA12, COMT) were over expressed in at least one LOAD cohort, and two were under expressed in LOAD (PDHB and RGS4). These constructs were replicated with the LOAD state, overexpressing HSPA2, GNA12, COMT and knocking down PDHB and RGS4. CCTS was not differentially expressed, but was followed as a replicated key driver peptide. CCTS was overexpressed as a first pass of replication. The goal was to obtain consistent measures of changes of Aβ and/or Tau at multiple time-points (48, 72 and 96 hours) after transduction.

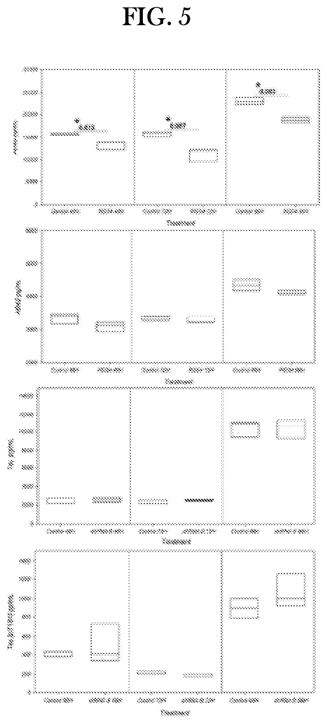

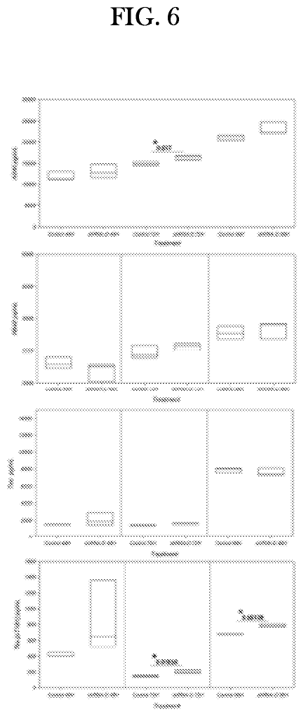

For RGS4, there was a significant downregulation of Abeta40 at all time points measured, but no effect on ABeta42 (FIG. 5). This drop in Aβ levels is counter to the effects seen in brain tissue. In the brains having less RGS4 was toxic and therefore, Aβ should be increased with less RGS4. Alternatively, the lower levels of RGS4 could be reflecting end-stage protective compensatory mechanisms, and in that context the result makes sense. The Aβ results for PDHB were for the most part non-significant, with only one time-point showing a difference in ABeta40 (FIG. 6). For Tau, there were no significant results with RGS4 nor was there any trend in the data (FIG. 6). For PDHB, there was no change in total Tau and Phospho-Tau was significant at two out of three time points measured (FIG. 6). These effects are consistent with the brain tissue data, since there was less expression of PDHB in LOAD brains therefore Tau should be increased with knockdown.

### EXAMPLE 2

### Insulin Sensitivity Measurement And IPSC Generation

The systems and methods of the present invention are also applicable to predictive network modeling in human induced pluripotent stem cells to identify key driver genes for insulin responsiveness.

Individuals in the study have accompanying genome-wide genotyping and gold standard measurement of insulin sensitivity (i.e. steady state plasma glucose-SSPG-derived from an insulin suppression test). Other biometric parameters include age, body mass index, sex and race/ethnicity (Table 1). Three to seven iPSC lines were generated from each individual, with no apparent differences in the reprogramming efficiency between IR and IS cells. The complete pipeline for iPSC generation and quality control has been previously described. Briefly, RNA-seq data for 317 ipSC lines from 101 individuals was generated and, after quality control, RNA-seq data from 310 samples from 100 individuals was analyzed, of which 48 were IS (149 samples) and 52 were IR (161 samples). The SSPG cut-off to discriminate between IR or IS state was set at 140 mg/dl based on previous publications. The average SSPG values were 84 mg/dl for the IS group and 210 mg/dl for the IR group. Finally, samples in both groups were age and body mass index (BMI) matched to avoid possible biases (mean age 57.7 years old and 59.5 in the IS vs. IR group, respectively, and mean BMI 28.5 in the IS vs. 30 in the IR group).

TABLE 1

| | | SSPG | | | | AGE | | BMI | |
|---|---|---|---|---|---|---|---|---|---|
| | n = | Min | Max | Average | Median | Average | SDEV | Average | SDEV |
| Insuin Sensitive | 49 | 41 | 136 | 84.2 | 81 | 57.5 | 8.4 | 28.4 | 3.7 |
| Insulin Resistant | 52 | 142 | 323 | 210 | 201 | 59.5 | 9 | 30 | 3.6 |

Differential Expression Analysis

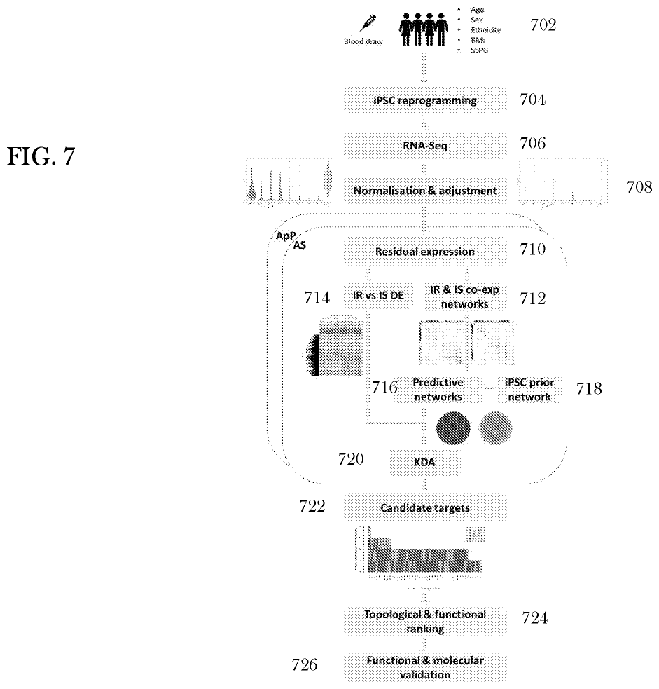

iPSC lines have been demonstrated to recapitulate many Mendelian diseases, including insulin resistance resulting from severe mutations in the insulin receptor. However, the extent to which iPSCs recapitulate the genetic architecture of common polygenic susceptibility to insulin sensitivity/resistance is unknown. As the holistic approach to compare IR and IS states was based and scaled to the full transcriptional network (FIG. 7), differential expression analysis was used to corroborate the hypothesis that iPSCs maintain components of the genetic architecture associated with the insulin sensitivity status of the individual donor. To that end, initially, there was a check of the 100 most significant DE genes between IR and IS iPSCs for enrichment of KEGG pathways. Among the top 10 most enriched pathways are sugar metabolism, insulin signaling, glycolysis/gluconeogenesis, and type II diabetes mellitus (Table 2), which suggests that iPSCs themselves reflect, at least in part, the insulin sensitivity status and molecular regulatory mechanisms of the individual donors.

TABLE 2

| pathway | pvalue | fdr |
|---|---|---|
| Neomycin, kanamycin and gentamicin biosynthesis—Homo sapiens | 7.81E−05 | 0.02570879 |
| (human) | | |
| Amino sugar and nucleotide sugar metabolism—Homo sapiens | 0.00132764 | 0.21839648 |
| (human) | | |
| Folate biosynthesis—Homo sapiens (human) | 0.00454363 | 0.32878345 |
| Galactose metabolism—Homo sapiens (human) | 0.00499671 | 0.32878345 |
| Starch and sucrose metabolism—Homo sapiens (human) | 0.00499671 | 0.32878345 |
| Alanine, aspartate and glutamate metabolism—Homo sapiens (human) | 0.00810328 | 0.44432985 |
| Arachidonic acid metabolism—Homo sapiens (human) | 0.01052114 | 0.49449368 |
| Type II diabetes mellitus—Homo sapiens (human) | 0.01535528 | 0.63148577 |
| Glycolysis / Gluconeogenesis—Homo sapiens (human) | 0.0200478 | 0.66373722 |
| Insulin signaling pathway—Homo sapiens (human) | 0.02017438 | 0.66373722 |
| Taurine and hypotaurine metabolism—Homo sapiens (human) | 0.03013285 | 0.86848501 |
| Central carbon metabolism in cancer—Homo sapiens (human) | 0.03167726 | 0.86848501 |
| C-type lectin receptor signaling pathway—Homo sapiens (human) | 0.05029528 | 1 |
| Phototransduction—Homo sapiens (human) | 0.05845049 | 1 |
| Measles—Homo sapiens (human) | 0.06151679 | 1 |
| Maturity onset diabetes of the young—Homo sapiens (human) | 0.0675004 | 1 |
| Steroid biosynthesis—Homo sapiens (human) | 0.08072037 | 1 |
| Butanoate metabolism—Homo sapiens (human) | 0.08503382 | 1 |
| Type I diabetes mellitus—Homo sapiens (human) | 0.10183106 | 1 |
| Carbohydrate digestion and absorption—Homo sapiens (human) | 0.10183106 | 1 |
| Calcium signaling pathway—Homo sapiens (human) | 0.10426845 | 1 |
| beta-Alanine metabolism—Homo sapiens (human) | 0.10591797 | 1 |
| Fructose and mannose metabolism—Homo sapiens (human) | 0.11791439 | 1 |
| Pathways in cancer—Homo sapiens (human) | 0.11911797 | 1 |
| Chemical carcinogenesis—Homo sapiens (human) | 0.12569515 | 1 |
| Metabolism of xenobiotics by cytochrome P450—Homo sapiens | 0.14074811 | 1 |
| (human) | | |
| Complement and coagulation cascades—Homo sapiens (human) | 0.14440731 | 1 |
| Axon guidance—Homo sapiens (human) | 0.14877009 | 1 |
| Cholesterol metabolism—Homo sapiens (human) | 0.15514032 | 1 |
| Cocaine addiction—Homo sapiens (human) | 0.15514032 | 1 |
| Arginine and proline metabolism—Homo sapiens (human) | 0.17553396 | 1 |
| Fanconi anemia pathway—Homo sapiens (human) | 0.20059771 | 1 |
| Lysine degradation—Homo sapiens (human) | 0.21225991 | 1 |
| Prolactin signaling pathway—Homo sapiens (human) | 0.21508795 | 1 |
| Endocytosis—Homo sapiens (human) | 0.21647316 | 1 |
| Human papillomavirus infection—Homo sapiens (human) | 0.23330493 | 1 |
| GABAergic synapse—Homo sapiens (human) | 0.23393482 | 1 |
| Insulin secretion—Homo sapiens (human) | 0.23902251 | 1 |
| p53 signaling pathway—Homo sapiens (human) | 0.24881535 | 1 |
| TGF-beta signaling pathway—Homo sapiens (human) | 0.25583326 | 1 |
| PI3K-Akt signaling pathway—Homo sapiens (human) | 0.25785212 | 1 |
| RNA degradation—Homo sapiens (human) | 0.26036004 | 1 |
| Ribosome biogenesis in eukaryotes—Homo sapiens (human) | 0.26257862 | 1 |
| Peroxisome—Homo sapiens (human) | 0.26257862 | 1 |
| T cell receptor signaling pathway—Homo sapiens (human) | 0.26692737 | 1 |
| Fc gamma R-mediated phagocytosis—Homo sapiens (human) | 0.26692737 | 1 |
| Glucagon signaling pathway—Homo sapiens (human) | 0.26692737 | 1 |
| ErbB signaling pathway—Homo sapiens (human) | 0.26905801 | 1 |
| MAPK signaling pathway—Homo sapiens (human) | 0.27609795 | 1 |
| HIF-1 signaling pathway—Homo sapiens (human) | 0.28124457 | 1 |
| Toxoplasmosis—Homo sapiens (human) | 0.28696425 | 1 |
| Insulin resistance—Homo sapiens (human) | 0.30270128 | 1 |
| NOD-like receptor signaling pathway—Homo sapiens (human) | 0.32191924 | 1 |
| Neuroactive ligand-receptor interaction—Homo sapiens (human) | 0.32323642 | 1 |
| Lysosome—Homo sapiens (human) | 0.32323642 | 1 |
| Tuberculosis—Homo sapiens (human) | 0.32828658 | 1 |
| HTLV-I infection—Homo sapiens (human) | 0.33705243 | 1 |
| Transcriptional misregulation in cancer—Homo sapiens (human) | 0.33737602 | 1 |
| Parkinson disease—Homo sapiens (human) | 0.34045186 | 1 |
| cGMP-PKG signaling pathway—Homo sapiens (human) | 0.3414382 | 1 |
| Thermogenesis—Homo sapiens (human) | 0.34239463 | 1 |
| Ubiquitin mediated proteolysis—Homo sapiens (human) | 0.34608552 | 1 |
| mTOR signaling pathway—Homo sapiens (human) | 0.34781482 | 1 |
| MicroRNAs in cancer—Homo sapiens (human) | 0.35105835 | 1 |
| Herpes simplex infection—Homo sapiens (human) | 0.35182535 | 1 |
| Purine metabolism—Homo sapiens (human) | 0.35857229 | 1 |
| Huntington disease—Homo sapiens (human) | 0.36777955 | 1 |
| Ras signaling pathway—Homo sapiens (human) | 0.40062765 | 1 |
| Human cytomegalovirus infection—Homo sapiens (human) | 0.40272834 | 1 |
| Epstein-Barr virus infection—Homo sapiens (human) | 0.40483989 | 1 |
| Focal adhesion—Homo sapiens (human) | 0.41124029 | 1 |
| Regulation of actin cytoskeleton—Homo sapiens (human) | 0.41774047 | 1 |
| Proteoglycans in cancer—Homo sapiens (human) | 0.42434196 | 1 |
| Rap1 signaling pathway—Homo sapiens (human) | 0.42880001 | 1 |
| Protein processing in endoplasmic reticulum—Homo sapiens | 0.44709882 | 1 |
| (human) | | |
| Viral carcinogenesis—Homo sapiens (human) | 0.44709882 | 1 |
| Alzheimer disease—Homo sapiens (human) | 0.45179257 | 1 |
| RNA transport—Homo sapiens (human) | 0.47105849 | 1 |
| cAMP signaling pathway—Homo sapiens (human) | 0.47600022 | 1 |
| Hepatocellular carcinoma—Homo sapiens (human) | 0.48099308 | 1 |
| Cellular senescence—Homo sapiens (human) | 0.48350884 | 1 |
| Tight junction—Homo sapiens (human) | 0.48350884 | 1 |
| Kaposi sarcoma-associated herpesvirus infection—Homo sapiens | 0.48350884 | 1 |
| (human) | | |
| Hippo signaling pathway—Homo sapiens (human) | 0.51205341 | 1 |
| Non-alcoholic fatty liver disease (NAFLD)—Homo sapiens (human) | 0.51205341 | 1 |
| Ribosome—Homo sapiens (human) | 0.51741903 | 1 |
| Spliceosome—Homo sapiens (human) | 0.5201226 | 1 |
| Oxytocin signaling pathway—Homo sapiens (human) | 0.52831719 | 1 |
| Autophagy—animal—Homo sapiens (human) | 0.53107692 | 1 |
| Oxidative phosphorylation—Homo sapiens (human) | 0.53663909 | 1 |
| Wnt signaling pathway—Homo sapiens (human) | 0.53944168 | 1 |
| Retrograde endocannabinoid signaling—Homo sapiens (human) | 0.54225871 | 1 |
| Cell cycle—Homo sapiens (human) | 0.54509025 | 1 |
| Hepatitis B—Homo sapiens (human) | 0.54509025 | 1 |
| Chemokine signaling pathway—Homo sapiens (human) | 0.54793637 | 1 |
| Phospholipase D signaling pathway—Homo sapiens (human) | 0.54793637 | 1 |
| Cushing syndrome—Homo sapiens (human) | 0.55079714 | 1 |
| Influenza A—Homo sapiens (human) | 0.55079714 | 1 |
| Apoptosis—Homo sapiens (human) | 0.55946815 | 1 |
| Signaling pathways regulating pluripotency of stem cells— | 0.55946815 | 1 |
| Homo sapiens (human) | | |
| Breast cancer—Homo sapiens (human) | 0.55946815 | 1 |
| Adrenergic signaling in cardiomyocytes—Homo sapiens (human) | 0.5623883 | 1 |
| Fluid shear stress and atherosclerosis—Homo sapiens (human) | 0.5623883 | 1 |
| Apelin signaling pathway—Homo sapiens (human) | 0.56532348 | 1 |
| Gastric cancer—Homo sapiens (human) | 0.56532348 | 1 |
| Cytokine-cytokine receptor interaction—Homo sapiens (human) | 0.56827376 | 1 |
| FoxO signaling pathway—Homo sapiens (human) | 0.56827376 | 1 |
| Necroptosis—Homo sapiens (human) | 0.57421997 | 1 |
| Relaxin signaling pathway—Homo sapiens (human) | 0.57421997 | 1 |
| Neurotrophin signaling pathway—Homo sapiens (human) | 0.57721604 | 1 |
| Sphingolipid signaling pathway—Homo sapiens (human) | 0.58325452 | 1 |
| AMPK signaling pathway—Homo sapiens (human) | 0.58325452 | 1 |
| Dopaminergic synapse—Homo sapiens (human) | 0.58325452 | 1 |
| Phagosome—Homo sapiens (human) | 0.58935529 | 1 |
| Thyroid hormone signaling pathway—Homo sapiens (human) | 0.58935529 | 1 |
| Hepatitis C—Homo sapiens (human) | 0.60174627 | 1 |
| Oocyte meiosis—Homo sapiens (human) | 0.60488394 | 1 |
| Estrogen signaling pathway—Homo sapiens (human) | 0.61439411 | 1 |
| Alcoholism—Homo sapiens (human) | 0.61439411 | 1 |
| Cell adhesion molecules (CAMs)—Homo sapiens (human) | 0.620816 | 1 |
| Platelet activation—Homo sapiens (human) | 0.62405173 | 1 |
| JAK-STAT signaling pathway—Homo sapiens (human) | 0.62405173 | 1 |
| Glutamatergic synapse—Homo sapiens (human) | 0.63057316 | 1 |
| Cholinergic synapse—Homo sapiens (human) | 0.63057316 | 1 |
| TNF signaling pathway—Homo sapiens (human) | 0.63385904 | 1 |
| Osteoclast differentiation—Homo sapiens (human) | 0.6371618 | 1 |
| Parathyroid hormone synthesis, secretion and action—Homo sapiens | 0.6371618 | 1 |
| (human) | | |
| AGE-RAGE signaling pathway in diabetic complications— | 0.6371618 | 1 |
| Homo sapiens (human) | | |
| Prostate cancer—Homo sapiens (human) | 0.6371618 | 1 |
| Pyrimidine metabolism—Homo sapiens (human) | 0.64048153 | 1 |
| Phosphatidylinositol signaling system—Homo sapiens (human) | 0.64048153 | 1 |
| Vascular smooth muscle contraction—Homo sapiens (human) | 0.64048153 | 1 |
| Leukocyte transendothelial migration—Homo sapiens (human) | 0.64048153 | 1 |
| Small cell lung cancer—Homo sapiens (human) | 0.64381833 | 1 |
| Choline metabolism in cancer—Homo sapiens (human) | 0.64381833 | 1 |
| mRNA surveillance pathway—Homo sapiens (human) | 0.65733782 | 1 |
| Colorectal cancer—Homo sapiens (human) | 0.65733782 | 1 |
| Longevity regulating pathway—Homo sapiens (human) | 0.66420218 | 1 |
| Progesterone-mediated oocyte maturation—Homo sapiens (human) | 0.66420218 | 1 |
| Th17 cell differentiation—Homo sapiens (human) | 0.67113724 | 1 |
| Glycerophospholipid metabolism—Homo sapiens (human) | 0.6746315 | 1 |
| GnRH signaling pathway—Homo sapiens (human) | 0.6746315 | 1 |
| Melanogenesis—Homo sapiens (human) | 0.6746315 | 1 |
| Serotonergic synapse—Homo sapiens (human) | 0.6781437 | 1 |
| Circadian entrainment—Homo sapiens (human) | 0.68167394 | 1 |
| Aldosterone synthesis and secretion—Homo sapiens (human) | 0.68167394 | 1 |
| Inflammatory mediator regulation of TRP channels— | 0.68878887 | 1 |
| Homo sapiens (human) | | |
| Chagas disease (American trypanosomiasis)—Homo sapiens (human) | 0.68878887 | 1 |
| Gap junction—Homo sapiens (human) | 0.69237376 | 1 |
| Pancreatic cancer—Homo sapiens (human) | 0.69237376 | 1 |
| Adherens junction—Homo sapiens (human) | 0.69597704 | 1 |
| Chronic myeloid leukemia—Homo sapiens (human) | 0.69597704 | 1 |
| Bacterial invasion of epithelial cells—Homo sapiens (human) | 0.70323919 | 1 |
| Natural killer cell mediated cytotoxicity—Homo sapiens (human) | 0.70689824 | 1 |
| Renal cell carcinoma—Homo sapiens (human) | 0.71057606 | 1 |
| Inositol phosphate metabolism—Homo sapiens (human) | 0.71427276 | 1 |
| Th1 and Th2 cell differentiation—Homo sapiens (human) | 0.71427276 | 1 |
| Amoebiasis—Homo sapiens (human) | 0.71427276 | 1 |
| Dilated cardiomyopathy (DCM)—Homo sapiens (human) | 0.71798842 | 1 |
| NF-kappa B signaling pathway—Homo sapiens (human) | 0.72172315 | 1 |
| ECM-receptor interaction—Homo sapiens (human) | 0.72172315 | 1 |
| Morphine addiction—Homo sapiens (human) | 0.72172315 | 1 |
| Salmonella infection—Homo sapiens (human) | 0.72172315 | 1 |
| Mitophagy—animal—Homo sapiens (human) | 0.72547704 | 1 |
| Glioma—Homo sapiens (human) | 0.72547704 | 1 |
| Non-small cell lung cancer—Homo sapiens (human) | 0.72925018 | 1 |
| Cardiac muscle contraction—Homo sapiens (human) | 0.73304267 | 1 |
| Hypertrophic cardiomyopathy (HCM)—Homo sapiens (human) | 0.73304267 | 1 |
| B cell receptor signaling pathway—Homo sapiens (human) | 0.73685462 | 1 |
| Protein digestion and absorption—Homo sapiens (human) | 0.73685462 | 1 |
| Epithelial cell signaling in Helicobacter pylori infection— | 0.73685462 | 1 |
| Homo sapiens (human) | | |
| Arrhythmogenic right ventricular cardiomyopathy (ARVC)— | 0.73685462 | 1 |
| Homo sapiens (human) | | |
| Toll-like receptor signaling pathway—Homo sapiens (human) | 0.74068612 | 1 |
| Shigellosis—Homo sapiens (human) | 0.74068612 | 1 |
| IL-17 signaling pathway—Homo sapiens (human) | 0.74453726 | 1 |
| Pancreatic secretion—Homo sapiens (human) | 0.74453726 | 1 |
| Long-term potentiation—Homo sapiens (human) | 0.74840816 | 1 |
| Adipocytokine signaling pathway—Homo sapiens (human) | 0.74840816 | 1 |
| Endometrial cancer—Homo sapiens (human) | 0.74840816 | 1 |
| Longevity regulating pathway—multiple species—Homo sapiens | 0.7522989 | 1 |
| (human) | | |
| Melanoma—Homo sapiens (human) | 0.7522989 | 1 |
| Thyroid hormone synthesis—Homo sapiens (human) | 0.75620959 | 1 |
| Salivary secretion—Homo sapiens (human) | 0.75620959 | 1 |
| Gastric acid secretion—Homo sapiens (human) | 0.75620959 | 1 |
| Acute myeloid leukemia—Homo sapiens (human) | 0.76014033 | 1 |
| Rheumatoid arthritis—Homo sapiens (human) | 0.76014033 | 1 |
| VEGF signaling pathway—Homo sapiens (human) | 0.76409122 | 1 |
| Long-term depression—Homo sapiens (human) | 0.76806236 | 1 |
| PPAR signaling pathway—Homo sapiens (human) | 0.77205386 | 1 |
| Synaptic vesicle cycle—Homo sapiens (human) | 0.77205386 | 1 |
| Amphetamine addiction—Homo sapiens (human) | 0.77205386 | 1 |
| Fc epsilon RI signaling pathway—Homo sapiens (human) | 0.77606581 | 1 |
| Pertussis—Homo sapiens (human) | 0.77606581 | 1 |
| N-Glycan biosynthesis—Homo sapiens (human) | 0.78009833 | 1 |
| Basal cell carcinoma—Homo sapiens (human) | 0.78009833 | 1 |
| Glycerolipid metabolism—Homo sapiens (human) | 0.78822546 | 1 |
| Cortisol synthesis and secretion—Homo sapiens (human) | 0.78822546 | 1 |
| Leishmaniasis—Homo sapiens (human) | 0.78822546 | 1 |
| Antigen processing and presentation—Homo sapiens (human) | 0.79232028 | 1 |
| RIG-I-like receptor signaling pathway—Homo sapiens (human) | 0.79232028 | 1 |
| Hematopoietic cell lineage—Homo sapiens (human) | 0.79232028 | 1 |
| Vibrio cholerae infection—Homo sapiens (human) | 0.79232028 | 1 |
| Aminoacyl-tRNA biosynthesis—Homo sapiens (human) | 0.79643608 | 1 |
| Notch signaling pathway—Homo sapiens (human) | 0.79643608 | 1 |
| Renin secretion—Homo sapiens (human) | 0.79643608 | 1 |
| Pathogenic Escherichia coli infection—Homo sapiens (human) | 0.79643608 | 1 |
| Valine, leucine and isoleucine degradation—Homo sapiens (human) | 0.80057297 | 1 |
| Drug metabolism—other enzymes—Homo sapiens (human) | 0.80057297 | 1 |
| Glutathione metabolism—Homo sapiens (human) | 0.80473105 | 1 |
| Nucleotide excision repair—Homo sapiens (human) | 0.80473105 | 1 |
| Amyotrophic lateral sclerosis (ALS)—Homo sapiens (human) | 0.80473105 | 1 |
| Proteasome—Homo sapiens (human) | 0.80891043 | 1 |
| Hedgehog signaling pathway—Homo sapiens (human) | 0.80891043 | 1 |
| Viral myocarditis—Homo sapiens (human) | 0.81311122 | 1 |
| Homologous recombination—Homo sapiens (human) | 0.81733352 | 1 |
| Regulation of lipolysis in adipocytes—Homo sapiens (human) | 0.81733352 | 1 |
| Bile secretion—Homo sapiens (human) | 0.81733352 | 1 |
| Systemic lupus erythematosus—Homo sapiens (human) | 0.81733352 | 1 |
| Sphingolipid metabolism—Homo sapiens (human) | 0.82157745 | 1 |
| Vasopressin-regulated water reabsorption—Homo sapiens (human) | 0.82157745 | 1 |
| Mineral absorption—Homo sapiens (human) | 0.82157745 | 1 |
| Endocrine and other factor-regulated calcium reabsorption— | 0.8258431 | 1 |
| Homo sapiens (human) | | |
| Legionellosis—Homo sapiens (human) | 0.8301306 | 1 |
| Bladder cancer—Homo sapiens (human) | 0.8301306 | 1 |
| Cysteine and methionine metabolism—Homo sapiens (human) | 0.83444006 | 1 |
| Basal transcription factors—Homo sapiens (human) | 0.83444006 | 1 |
| DNA replication—Homo sapiens (human) | 0.83444006 | 1 |
| Ferroptosis—Homo sapiens (human) | 0.83444006 | 1 |
| Fatty acid degradation—Homo sapiens (human) | 0.84312527 | 1 |
| Base excision repair—Homo sapiens (human) | 0.84312527 | 1 |
| Cytosolic DNA-sensing pathway—Homo sapiens (human) | 0.84312527 | 1 |
| Thyroid cancer—Homo sapiens (human) | 0.84312527 | 1 |
| Pyruvate metabolism—Homo sapiens (human) | 0.84750125 | 1 |
| Ether lipid metabolism—Homo sapiens (human) | 0.85189962 | 1 |
| SNARE interactions in vesicular transport—Homo sapiens (human) | 0.85189962 | 1 |
| Autophagy other—Homo sapiens (human) | 0.85189962 | 1 |
| Taste transduction—Homo sapiens (human) | 0.85189962 | 1 |
| Inflammatory bowel disease (IBD)—Homo sapiens (human) | 0.85189962 | 1 |
| Glycine, serine and threonine metabolism—Homo sapiens (human) | 0.85632052 | 1 |
| Ovarian steroidogenesis—Homo sapiens (human) | 0.85632052 | 1 |
| Fatty acid elongation—Homo sapiens (human) | 0.86076403 | 1 |
| RNA polymerase—Homo sapiens (human) | 0.86076403 | 1 |
| Apoptosis—multiple species—Homo sapiens (human) | 0.86076403 | 1 |
| Citrate cycle (TCA cycle)—Homo sapiens (human) | 0.86523029 | 1 |
| Propanoate metabolism—Homo sapiens (human) | 0.86523029 | 1 |
| Circadian rhythm—Homo sapiens (human) | 0.86523029 | 1 |
| Aldosterone-regulated sodium reabsorption—Homo sapiens (human) | 0.86523029 | 1 |
| Tryptophan metabolism—Homo sapiens (human) | 0.8697194 | 1 |
| Drug metabolism—cytochrome P450—Homo sapiens (human) | 0.8697194 | 1 |
| ABC transporters—Homo sapiens (human) | 0.8697194 | 1 |
| Hippo signaling pathway—multiple species—Homo sapiens (human) | 0.8697194 | 1 |
| Olfactory transduction—Homo sapiens (human) | 0.8697194 | 1 |
| Glyoxylate and dicarboxylate metabolism—Homo sapiens (human) | 0.87423148 | 1 |
| Pentose phosphate pathway—Homo sapiens (human) | 0.88790672 | 1 |
| Mannose type O-glycan biosynthesis—Homo sapiens (human) | 0.88790672 | 1 |
| Glycosylphosphatidylinositol (GPI)—anchor biosynthesis— | 0.88790672 | 1 |
| Homo sapiens (human) | | |
| Nicotinate and nicotinamide metabolism—Homo sapiens (human) | 0.88790672 | 1 |
| Protein export—Homo sapiens (human) | 0.88790672 | 1 |
| Mucin type O-glycan biosynthesis—Homo sapiens (human) | 0.89251185 | 1 |
| Other types of O-glycan biosynthesis—Homo sapiens (human) | 0.89251185 | 1 |
| Porphyrin and chlorophyll metabolism—Homo sapiens (human) | 0.89251185 | 1 |
| Biosynthesis of unsaturated fatty acids—Homo sapiens (human) | 0.89251185 | 1 |
| Mismatch repair—Homo sapiens (human) | 0.89251185 | 1 |
| Intestinal immune network for IgA production—Homo sapiens | 0.89251185 | 1 |
| (human) | | |
| Glycosaminoglycan biosynthesis—heparan sulfate / heparin— | 0.89714053 | 1 |
| Homo sapiens (human) | | |
| Glycosphingolipid biosynthesis—lacto and neolacto series— | 0.89714053 | 1 |
| Homo sapiens (human) | | |
| Collecting duct acid secretion—Homo sapiens (human) | 0.89714053 | 1 |
| Retinol metabolism—Homo sapiens (human) | 0.90179289 | 1 |
| Terpenoid backbone biosynthesis—Homo sapiens (human) | 0.90179289 | 1 |
| Fat digestion and absorption—Homo sapiens (human) | 0.90179289 | 1 |
| Prion diseases—Homo sapiens (human) | 0.90179289 | 1 |
| Nicotine addiction—Homo sapiens (human) | 0.90179289 | 1 |
| Glycosaminoglycan biosynthesis—chondroitin sulfate / dermatan | 0.90646904 | 1 |
| sulfate—Homo sapiens (human) | | |
| One carbon pool by folate—Homo sapiens (human) | 0.90646904 | 1 |
| Staphylococcus aureus infection—Homo sapiens (human) | 0.90646904 | 1 |
| Proximal tubule bicarbonate reclamation—Homo sapiens (human) | 0.91116911 | 1 |
| Malaria—Homo sapiens (human) | 0.91116911 | 1 |
| Steroid hormone biosynthesis—Homo sapiens (human) | 0.91589321 | 1 |
| Arginine biosynthesis— Homo sapiens (human) | 0.91589321 | 1 |
| Tyrosine metabolism—Homo sapiens (human) | 0.91589321 | 1 |
| Selenocompound metabolism—Homo sapiens (human) | 0.91589321 | 1 |
| Autoimmune thyroid disease—Homo sapiens (human) | 0.91589321 | 1 |
| Other glycan degradation—Homo sapiens (human) | 0.92064147 | 1 |
| Glycosaminoglycan degradation—Homo sapiens (human) | 0.92064147 | 1 |
| African trypanosomiasis—Homo sapiens (human) | 0.92064147 | 1 |
| Allograft rejection—Homo sapiens (human) | 0.92064147 | 1 |
| Graft-versus-host disease—Homo sapiens (human) | 0.92064147 | 1 |
| Glycosphingolipid biosynthesis—ganglio series—Homo sapiens | 0.925414 | 1 |
| (human) | | |
| Vitamin digestion and absorption—Homo sapiens (human) | 0.925414 | 1 |
| Primary immunodeficiency—Homo sapiens (human) | 0.925414 | 1 |
| Histidine metabolism—Homo sapiens (human) | 0.93021094 | 1 |
| Glycosaminoglycan biosynthesis—keratan sulfate— | 0.93021094 | 1 |
| Homo sapiens (human) | | |
| Glycosphingolipid biosynthesis—globo and isoglobo series— | 0.93021094 | 1 |
| Homo sapiens (human) | | |
| Nitrogen metabolism—Homo sapiens (human) | 0.93503239 | 1 |
| Renin-angiotensin system—Homo sapiens (human) | 0.93503239 | 1 |
| Pentose and glucuronate interconversions—Homo sapiens (human) | 0.9398785 | 1 |
| alpha-Linolenic acid metabolism—Homo sapiens (human) | 0.9398785 | 1 |
| Thiamine metabolism—Homo sapiens (human) | 0.9398785 | 1 |
| Pantothenate and CoA biosynthesis—Homo sapiens (human) | 0.94474937 | 1 |
| Non-homologous end-joining—Homo sapiens (human) | 0.94474937 | 1 |
| Linoleic acid metabolism—Homo sapiens (human) | 0.94964514 | 1 |
| Fatty acid biosynthesis—Homo sapiens (human) | 0.95456593 | 1 |
| Primary bile acid biosynthesis—Homo sapiens (human) | 0.95456593 | 1 |
| Ubiquinone and other terpenoid-quinone biosynthesis—Homo sapiens | 0.95456593 | 1 |
| (human) | | |
| Phenylalanine metabolism—Homo sapiens (human) | 0.95456593 | 1 |
| Asthma—Homo sapiens (human) | 0.95456593 | 1 |
| Sulfur metabolism—Homo sapiens (human) | 0.95951187 | 1 |
| Sulfur relay system—Homo sapiens (human) | 0.95951187 | 1 |
| Ascorbate and aldarate metabolism—Homo sapiens (human) | 0.96448308 | 1 |
| Synthesis and degradation of ketone bodies—Homo sapiens (human) | 0.96448308 | 1 |
| Riboflavin metabolism—Homo sapiens (human) | 0.96448308 | 1 |
| Phosphonate and phosphinate metabolism—Homo sapiens (human) | 0.96947969 | 1 |
| Vitamin B6 metabolism—Homo sapiens (human) | 0.96947969 | 1 |
| D-Glutamine and D-glutamate metabolism—Homo sapiens (human) | 0.97450183 | 1 |
| Valine, leucine and isoleucine biosynthesis—Homo sapiens (human) | 0.98462322 | 1 |
| Phenylalanine, tyrosine and tryptophan biosynthesis—Homo sapiens | 0.98462322 | 1 |
| (human) | | |
| Biotin metabolism—Homo sapiens (human) | 0.98462322 | 1 |
| Lipoic acid metabolism—Homo sapiens (human) | 0.98462322 | 1 |
| Caffeine metabolism—Homo sapiens (human) | 0.98972272 | 1 |
| D-Arginine and D-ornithine metabolism—Homo sapiens (human) | 1 | 1 |
| Metabolic pathways—Homo sapiens (human) | 1 | 1 |
| Carbon metabolism—Homo sapiens (human) | 1 | 1 |
| 2-Oxocarboxylic acid metabolism—Homo sapiens (human) | 1 | 1 |
| Fatty acid metabolism—Homo sapiens (human) | 1 | 1 |
| Biosynthesis of amino acids—Homo sapiens (human) | 1 | 1 |
| EGFR tyrosine kinase inhibitor resistance—Homo sapiens (human) | 1 | 1 |
| Endocrine resistance—Homo sapiens (human) | 1 | 1 |
| Antifolate resistance—Homo sapiens (human) | 1 | 1 |
| Platinum drug resistance—Homo sapiens (human) | 1 | 1 |

FIG. 7 shows a flowchart that exemplarily delineates the logical flow of an exemplary embodiment of the invention. That logic flow may be implemented in software in certain embodiments of the invention. At step 702, blood is extracted from the target groups, and biometric parameters including age, body mass index, sex and race/ethnicity are collected. At step 704, “iPSC reprogramming,” the iPSCs are reprogrammed as explained in further detail herein. At step 706, “RNA-seq,” RNA sequencing is performed. At step 708, “Normalisation and adjustment,” the samples are normalized and adjusted.

Due to having multiple clones per patient, the effective sample size of the analyses is the number of patients, not the number of samples. However, since insulin resistance status is an individual-level characteristic, it could not adjust for donor without removing the signal of interest. Therefore, the data was analyzed in two ways. First, all samples were used (referred to as the AS analysis) without accounting for multiple clones for each individual. Second, the residual expression levels for all clones of each individual were averaged (referred to as the average-per-patient, ApP, analysis). While generalized estimating equations, mixed models or similar techniques can be used to compute residual expression levels taking into account the correlation of samples from the same individuals, it will not solve the problem of constructing co-expression and predictive networks for data with multiple clones per individual.

This analysis includes step 710, “Residual expression,” step 712, “IR & IS co-exp networks,” step 714, “IR vs IS DE,” step 716, “Predictive networks,” step 718, “iPSC prior network,” and step 720, “KDA.” Step 710 involves analysis of the residual expression of the genes. That analysis progresses to step 714, where AS and ApP DE analysis is performed as explained in the “Differential Expression Analysis” section below, and/or step 712, where co-expression analysis is performed, as explained in the “Co-Expression Network Analysis” section below. At step 716, “Predictive networks,” a predictive network analysis is performed, as explained in the “Predictive Network” section below. At step 718, “iPSC prior network,” the predictive network analysis incorporates prior network analyses that were performed in prior sample sets. At step 720, “KDA,” a Key Driver Analysis (KDA) is performed and the samples are ranked, as further explained in the “Key Driver Analysis (KDA) and Ranking” section below. Finally, using that data, at step 722, “Candidate targets,” target gene candidates are identified by the algorithm.

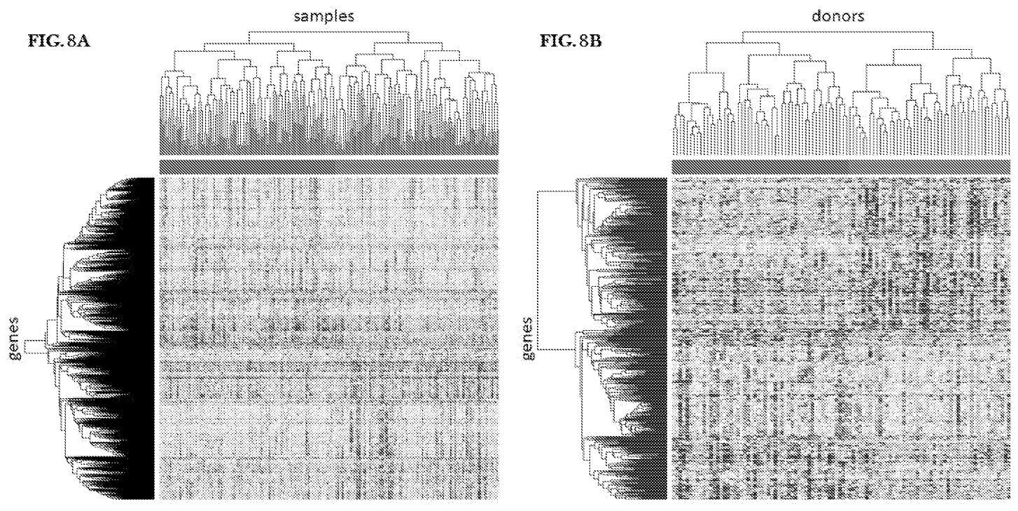

There were 1,338 genes identified that differentially expressed genes between IR and IS samples in the AS analysis (FDR adjusted p-value<0.05) whereas in the ApP analysis no significantly differentially expressed genes were identified. However, the comparison of the rank of the test statistics for the 500 most differentially expressed genes from both analyses shows consistent results, with very similar ranks for AS and ApP DE analysis (Spearman correlation coefficient=0.66, median rank in both AS and ApP=308, paired Wilcoxon signed-rank test P=0.90) (FIG. 8A-B). This result suggests that the lack of statistically significant DE genes in the ApP analysis is due to the reduced sample size. We therefore decided to use both the AS and ApP approaches, thereby consistently using the same residual expression levels in all analyses.

Co-Expression Network Analysis

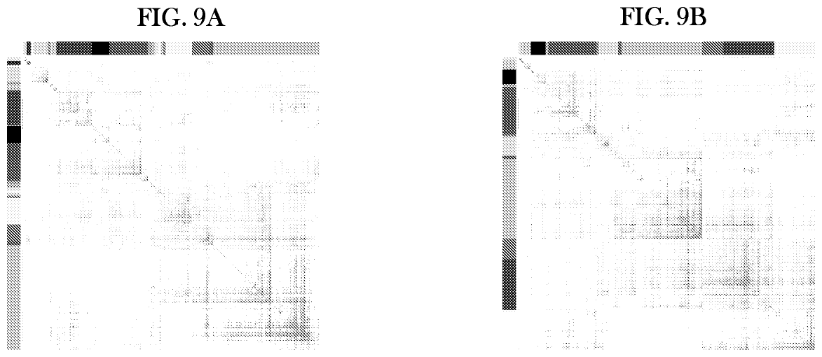

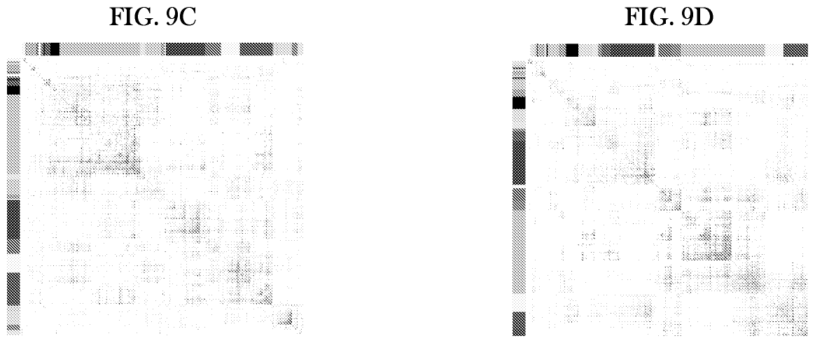

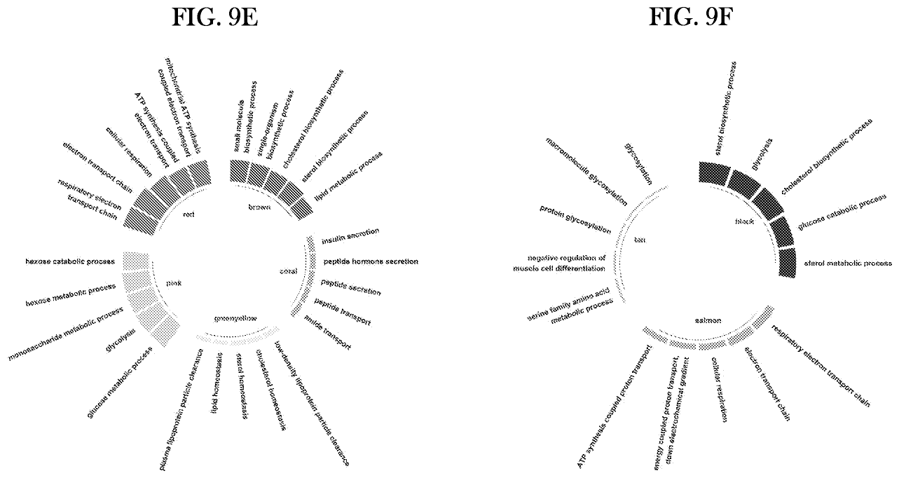

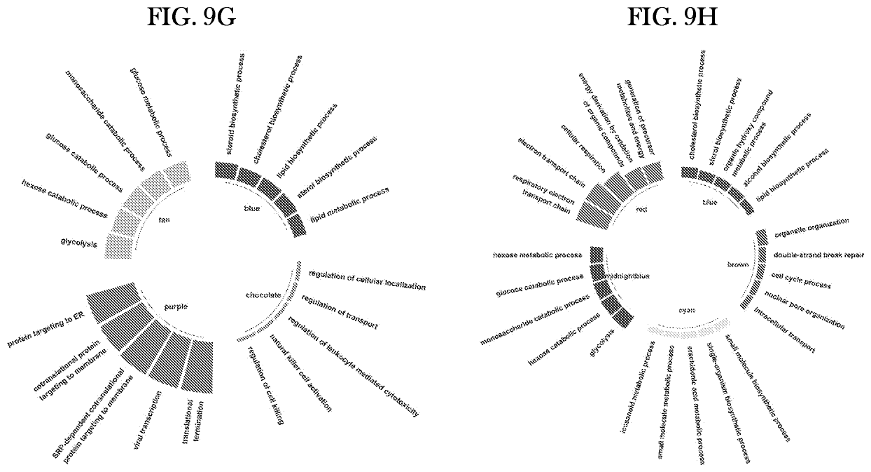

Four co-expression networks were trained in accordance with the present invention, as shown in FIG. 9A-D: for each of the AS and ApP adjusted expression residuals, one network was built for IR samples and another one for IS samples. Co-expression networks identify groups of genes (modules), with highly correlated expression patterns across samples indicating that they are involved in similar biological processes. A gene set enrichment analysis (GSEA) was used and the gene ontology (GO, C5 biological processes, v5.1) gene sets from the Molecular Signatures Database (MSigDB) were used to test the modules of each co-expression network for enrichment in insulin or metabolism related pathways. Specifically, 5 relevant modules in the AS IR network were identified (corresponding to a total of 1,565 genes), 3 modules were identified in the AS IS network (430 genes), 4 modules were identified in the ApP IR network (1,689 genes), and 5 modules were identified in the ApP IS network (2,791 genes). These modules are shown in FIG. 9E-H.

Predictive Networks

Seeding genes for each predictive network were obtained by expanding the set of genes in the selected modules from the corresponding co-expression network by including all genes connected to any of the selected module genes in k=3 or fewer steps in a prior, cell type-specific network derived from public gene and protein interaction databases: ConsensusPathDB (CPDB) and MetaCore (v6.24 from Thomson Reuters). The above seeding gene selection process ensures that genes impacting insulin sensitivity are included, while reducing the feature space by excluding irrelevant genes to train the predictive network. The final seeding gene sets consisted of 7,250 (AS IR), 3,797 (AS IS), 8,183 (ApP IR) and 9,712 (ApP IS) genes respectively. This final seeding gene list was used in the top-down and bottom-up predictive network pipeline (see online method) to build causal network models.

Key Driver Analysis (KDA) and Ranking

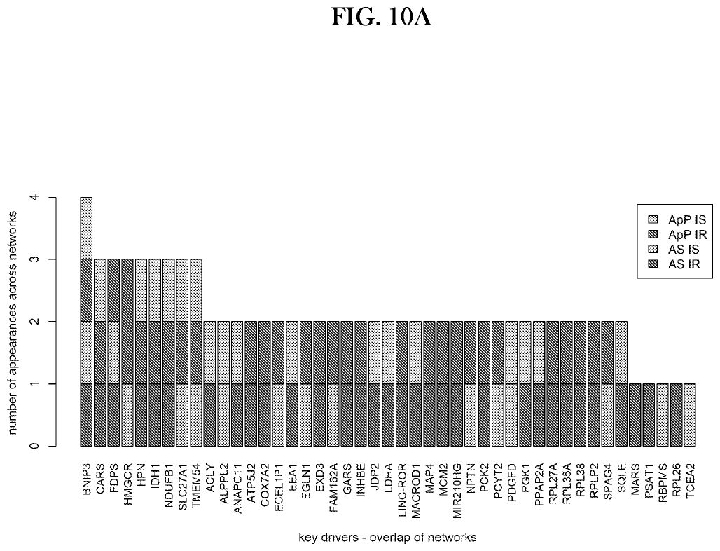

KDA was performed on the four predictive networks and a total of 281 key drivers (KD) in the AS predictive networks and 259 key drivers in the ApP networks were identified. There were 45 key driver genes common to both sets. KDA requires a starting set of genes to be specified and the KDA was run multiple times, once for the genes in each co-expression module that was enriched for pathways associated to insulin sensitivity as well as the DE genes from the AS and ApP analyses (5% FDR for AS and the top 500 DE genes for ApP). That means that a given gene can be identified as a KD for more than one set of genes (each representing different pathways) and the more often a gene is identified, the stronger the evidence it is implicated in the phenotype. There were 9 genes (BNIP3, CARS, IDH1, NDUFB1, HMGCR, HPN, FDPS, SLC27A1, TMEM54) identified that are KDs 3 or more times in the AS and ApP networks (FIGS. 10A-10B).

For these top 9 KDs, the topology of the local sub-networks around each of them was examined in the four networks. Specifically, 2 scores were computed (Table 3): the sum of the inverse path lengths from each key driver to the significantly differentially expressed genes in the AS networks and the top 500 most differentially expressed genes in the ApP network (DE proximity score) and second, the difference between the sums of the inverse path lengths from each KD to other KDs downstream of them and the inverse path lengths to other KDs upstream of them (KD dominance score). The higher the DE proximity score, the more there are paths from this KD to DE genes and/or the shorter these paths are; the higher the KD dominance score, the more other KDs are more directly downstream of this KD and/or the fewer other KDs are directly upstream of it.

Finally, a directed literature search was performed, combining the different KDs with the terms insulin, glucose, and diabetes. The results of the relevant publications associating the KDs to insulin sensitivity are summarized in Table 3 below. Briefly, both BNIP3 and SLC27A1 have been strongly associated with insulin resistance phenotypes. Solute Carrier Family 27 Member 1 (SLC27A1, also known as FATP1) is an insulin-sensitive fatty acid transporter involved in diet-induced obesity and has been associated to IR in skeletal muscle and BCL2/Adenovirus E1B 19kDa Interacting Protein 3 (BNIP3) is essential for mitochondrial bioenergetics during adipocyte remodeling, regulates mitochondrial function and lipid metabolism in the liver and in conjunction with PPARγcouples mitochondrial fusion-fission balance to systemic insulin sensitivity³⁰. Although informative about the quality of the KDA, the body of publications related to these two KDs decreased the interest on further validation. Among the remaining top KDs, HMGCR and FDPS have the highest combined DE proximity and KD dominance scores and both genes participate, together with squalene epoxidase (SQLE)—another KD that appears in both AS and ApP networks (Table 3)—in the cholesterol biosynthesis pathway. Given, i) the high DE proximity and KD dominance scores, ii) the shared metabolic pathway, iii) the widespread use of statins (HMGCR inhibitors) as therapeutic drugs to lower cholesterol levels in patients with high LDL-cholesterol, and iv) the emerging role of HMGCR in energy balance, metabolism and diabetes risk, validation of the KDA was sought, both transcriptionally and functionally, focusing on HMGCR, FDPS and SQLE.

TABLE 3

| | | Network | DE | KD |
|---|---|---|---|---|
| KD | GENE | appearances | proximity | dominance |
| BNIP3 | BCL2/Adenovirus | 4 | 20.30 | 0.00 |
| | E1B 19kDa | | | |
| | Interacting Protein 3 | | | |
| FDPS | Farnesyl Diphosphate | 3 | 16.22 | 1.42 |
| | Synthase | | | |
| SLC27A1 | Solute Carrier Family | 3 | 10.85 | 0.25 |
| | 27 Member 1 | | | |
| HMGCR | 3-Hydroxy-3- | 3 | 10.15 | 0.58 |
| | Methylglutaryl-CoA | | | |
| | Reductase | | | |
| CARS | Cysteinyl-TRNA | 3 | 7.80 | 0.00 |
| | Synthetase | | | |
| TMEM54 | Transmembrane | 3 | 7.57 | 1.00 |
| | Protein 54 | | | |
| IDH1 | Isocitrate | 3 | 7.32 | −3.25 |
| | Dehydrogenase | | | |
| | (NADP(+)) 1, | | | |
| | Cytosolic | | | |
| HPN | Hepsin | 3 | 6.67 | 0.00 |
| NDUFBI | NADH: | 3 | 6.45 | 0.00 |
| | Ubiquinone | | | |
| | Oxidoreductase | | | |
| | Subunit B1 | | | |

Table 3, above, shows summary statistics and references for the top ranked key drivers. Network appearances: number of appearances across IR and IS networks for AS and ApP DE proximity: sum of the inverse path lengths from each key driver to the significantly differentially expressed genes in the AS networks and the top 500 most differentially expressed genes in the ApP network. KD dominance: the difference between the sums of the inverse path lengths from each KD to other KDs downstream of them and the inverse path lengths to other KDs upstream of them. KD: key driver.

Network Validation

The causal IR/IS networks and the key driver analysis were validated in accordance with the present invention using the DE gene signature from an HMGCR inhibition experiment in iPSC cell lines derived from both IR (n=6) and IS individuals (n=6). For each iPSC line in this experiment (Table 4) RNA-seq data was generated in presence or absence of atorvastatin, an HMGCR inhibitor (statin) widely used in patients with hypercholesterolemia. Previous efforts have validated predictive networks through similar approaches.

TABLE 4

| Sample | Sample | | | | | | | | |
|---|---|---|---|---|---|---|---|---|---|
| ID | Name | Sample | State | Age | SEX | RACE | SSPG | BMI | Indiv |
| F9800001 | Genesips_1 | 255-1-D | Insulin resistant | 68 | Female | White.Hispanic | 323 | 27.4 | 255 |
| F9800002 | Genesips_2 | 255-1-A | Insulin resistant | 68 | Female | White.Hispanic | 323 | 27.4 | 255 |
| F9800003 | Genesips_3 | 243-1-D | Insulin resistant | 50 | Female | White | 209 | 34.7 | 243 |
| F9800004 | Genesips_4 | 243-1-A | Insulin resistant | 50 | Female | White | 209 | 34.7 | 243 |
| F9800005 | Genesips_5 | 688-1-D | Insulin resistant | 55 | Male | White | 279 | 33.1 | 688 |
| F9800006 | Genesips_6 | 688-1-A | Insulin resistant | 55 | Male | White | 279 | 33.1 | 688 |
| F9800007 | Genesips_7 | 108-2-D | Insulin sensitive | 49 | Male | White | 88 | 30.2 | 108 |
| F9800008 | Genesips_8 | 108-2-A | Insulin sensitive | 49 | Male | White | 88 | 30.2 | 108 |
| F9800009 | Genesips_9 | 193-4-D | Insulin resistant | 70 | Female | White | 255 | 27.5 | 193 |
| F9800010 | Genesips_10 | 193-4-A | Insulin resistant | 70 | Female | White | 255 | 27.5 | 193 |
| F9800011 | Genesips_11 | 420-1-D | Insulin sensitive | 70 | Male | White | 67 | 24.3 | 420 |
| F9800012 | Genesips_12 | 420-1-A | Insulin sensitive | 70 | Male | White | 67 | 24.3 | 420 |
| F9800013 | Genesips_13 | 336-3-D | Insulin resistant | 70 | Female | White | 243 | 33.2 | 335 |
| F9800014 | Genesips_14 | 336-3-A | Insulin resistant | 70 | Female | White | 243 | 33.2 | 335 |
| F9800015 | Genesips_15 | 239-3-D | Insulin resistant | 68 | Male | White | 272 | 34.3 | 239 |
| F9800016 | Genesips_16 | 239-3-A | Insulin resistant | 68 | Male | White | 272 | 34.3 | 239 |
| F9800017 | Genesips_17 | 541-2-D | Insulin sensitive | 64 | Male | White | 60 | 25.4 | 541 |
| F9800018 | Genesips_18 | 541-2-A | Insulin sensitive | 64 | Male | White | 60 | 25.4 | 541 |
| F9800019 | Genesips_19 | 475-1-D | Insulin sensitive | 41 | Male | White | 69 | 24.7 | 475 |
| F9800020 | Genesips_20 | 475-1-A | Insulin sensitive | 41 | Mele | White | 69 | 24.7 | 475 |
| F9800021 | Genesips_21 | 780-2-D | Insulin sensitive | 62 | Female | White | 82 | 21.9 | 780 |
| F9800022 | Genesips_22 | 780-2-A | Insulin sensitive | 62 | Female | White | 82 | 21.9 | 780 |
| F9800023 | Genesips_23 | 215-3-D | Insulin sensitive | 51 | Female | White | 91 | 23.6 | 215 |
| F9800024 | Genesips_24 | 215-3-A | Insulin sensitive | 51 | Female | White | 91 | 23.6 | 215 |

| Sample | | Sequencing | | | | |
|---|---|---|---|---|---|---|
| ID | totalReads | batch | Reprogramming.Source | Reprogramming.Batch | Sendal.Virus.Lot | RNA.Harvest.Date |
| F9800001 | | B243 | Activated T-Cells | 1-Jan | unknown | Sep. 12, 2017 |
| F9800002 | | B243 | Activated T-Cells | 1-Jan | unknown | Sep. 12, 2017 |
| F9800003 | | B243 | Erythroblasts | 1-80 | 1264008A | Sep. 12, 2017 |
| F9800004 | | B243 | Erythroblasts | 1-80 | 1264008A | Sep. 12, 2017 |
| F9800005 | | B243 | Erythroblasts | 1-100 | 1264008A | Sep. 12, 2017 |
| F9800006 | | B243 | Erythroblasts | 1-100 | 1264008A | Sep. 12, 2017 |
| F9800007 | | B243 | Erythroblasts | 1-80 | unknown | Sep. 12, 2017 |
| F9800008 | | B243 | Erythroblasts | 1-80 | unknown | Sep. 12, 2017 |
| F9800009 | | B243 | Erythroblasts | 1-70 | 1264008A | Sep. 12, 2017 |
| F9800010 | | B243 | Erythroblasts | 1-70 | 1264008A | Sep. 12, 2017 |
| F9800011 | | L222 | Erythroblasts | 1-40 | 1264008A | Sep. 12, 2017 |
| F9800012 | | L222 | Erythroblasts | 1-40 | 1264008A | Sep. 12, 2017 |
| F9800013 | | L222 | Erythroblasts | 1-80 | 1264008A | Sep. 12, 2017 |
| F9800014 | | L222 | Erythroblasts | 1-80 | 1264008A | Sep. 12, 2017 |
| F9800015 | | L222 | Erythroblasts | 1-90 | 1264008A | Sep. 16, 2017 |
| F9800018 | | L222 | Erythroblasts | 1-90 | 1264008A | Sep. 16, 2017 |
| F9800017 | | L222 | Erythroblasts | 1-110 | 1264014A | Sep. 16, 2017 |
| F9800018 | | L222 | Erythroblasts | 1-110 | 1264014A | Sep. 16, 2017 |
| F9800019 | | L222 | Erythroblasts | 1-60 | 1264008A | Sep. 24, 2017 |
| F9800020 | | L222 | Erythroblasts | 1-60 | 1264008A | Sep. 24, 2017 |
| F9800021 | | L222 | Erythroblasts | 1-70 | 1264008A | Sep. 24, 2017 |
| F9800022 | | L222 | Erythroblasts | 1-70 | 1264008A | Sep. 24, 2017 |
| F9800023 | | L222 | Erythroblasts | 1-50 | 1264008A | Sep. 24, 2017 |
| F9800024 | | L222 | Erythroblasts | 1-50 | 1264008A | Sep. 24, 2017 |

The comparison of atorvastatin-treated and untreated samples resulted in a list of 3205 DE genes that showed the highest enrichment for statin action pathway and cholesterol biosynthetic pathway and other related pathways, suggesting that HMGCR inhibition triggers a transcriptional compensatory response to balance the decrease in the cholesterol pathway intermediates.

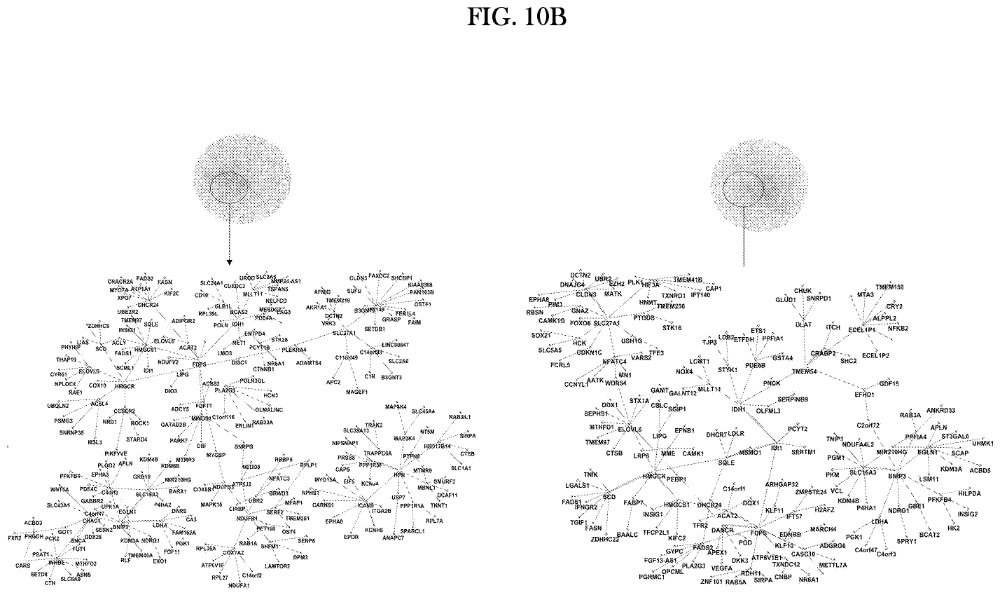

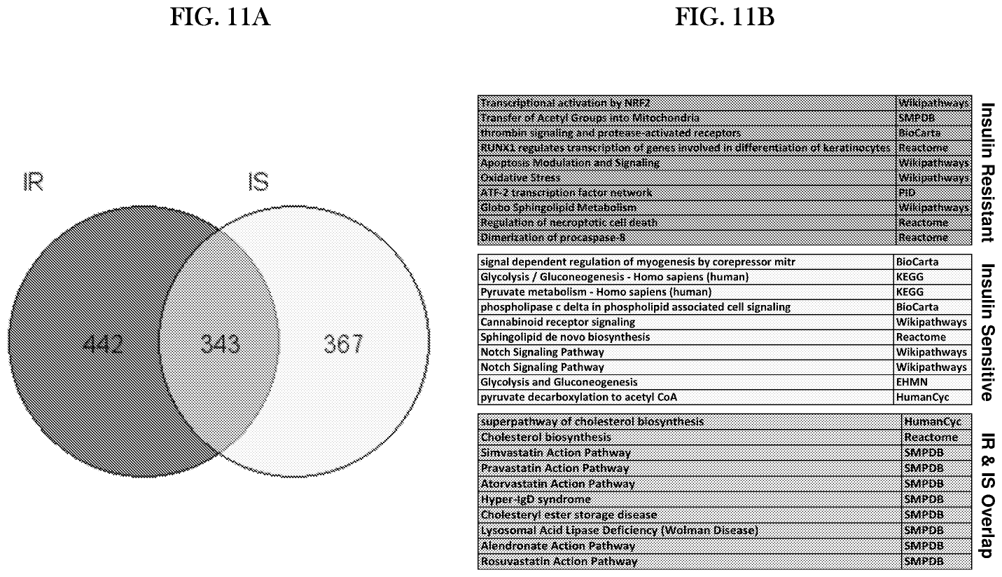

Next, re the specific responses of IR vs. IS iPSCs was compared to atorvastatin treatment. When considering IR or IS groups independently the number of DE genes was reduced to 785 (IR) and 711 (IS). As shown in FIG. 11A, DE genes between IR and IS samples are just partially overlapping (343/785 or 343/711, respectively), which suggests a differential response to atorvastatin treatment based on the IR/IS status of the donors. Moreover, pathway enrichment analysis for the 442 IR-specific and 367 IS-specific DE genes demonstrated striking differences in the enriched pathways between IR and IS iPSCs (FIG. 11B), which could be related to the disproportionate incidence of T2D in insulin resistant patients under statin treatment. Although atorvastatin treatment translates the perturbation of a metabolic pathway into measurable transcriptional changes and gives clues about HMGCR functionality and its association to insulin sensitivity, it requires of novel additional analyses to validate the predictive network.

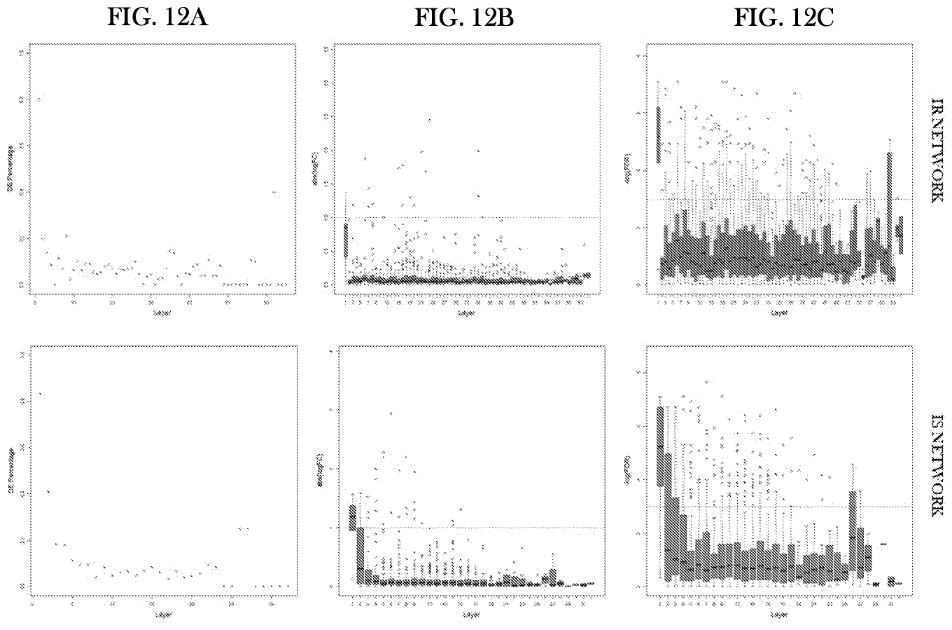

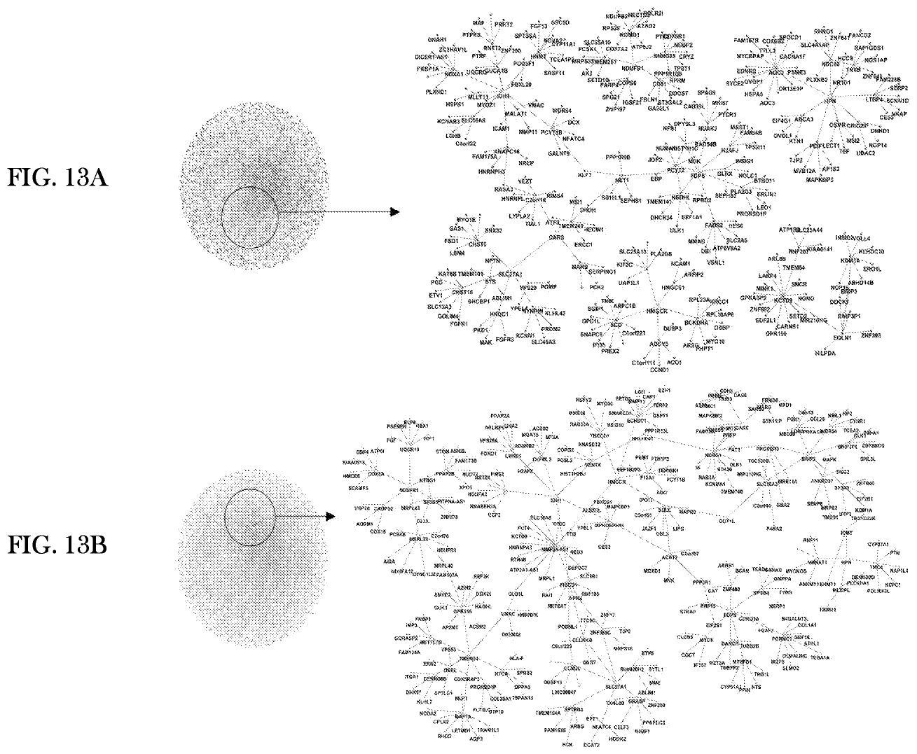

Validation of the network structure preferably involves multiple steps in certain embodiments of the invention: (1) calculation of the percentage of genes in each downstream layer of HMGCR in the network that are significantly altered (FDR<0.05) in gene expression in HMGCR inhibition experiment. It was found that for both IR and IS networks more than 80% of the genes in the first layer downstream of HMGCR are DE genes and that this percentage decreases as the distance to HMGCR increases in the network (FIG. 12A); and (2) comparison of DE gene fold-changes (log FC) (FIG. 12B) and associated significance (−log FDR) (FIG. 12C) to the AS network topology. Among the genes located downstream of HMGCR in both the IR and IS networks, the fold change and their significance decreases as the distance to HMGCR increases (FIG. 12B, FIG. 12C). The same pattern was observed in the ApP network (FIGS. 13A-13B). Finally, all four predictive networks (AS IR, AS IS, ApP IR and ApP IS) show a significant enrichment downstream of HMGCR for DE genes due to HMGCR inhibition (Table 5).

TABLE 5

| | AS—IR | AS—IS | ApP—IR | ApP—IS |
|---|---|---|---|---|
| number of DE genes due to HMGCR inhibition | 785 | 710 | 785 | 710 |
| number of genes not DE after HMGCR | 14339 | 14298 | 14506 | 14652 |
| inhibition | | | | |
| number of DE genes downstream | 164 | 155 | 297 | 302 |
| of HMGCR in the predictive network | | | | |
| number of DE genes not downstream | 621 | 555 | 488 | 408 |
| of HMGCR in the predictive network | | | | |
| number of non-DE genes downstream | 2357 | 1835 | 4634 | 5041 |
| of HMGCR in the predictive network | | | | |
| number of non-DE genes not downstream | 11982 | 12463 | 9872 | 9611 |
| of HMGCR in the predictive network | | | | |
| enrichment fold (if given as the risk ratio) | 1.27 | 1.70 | 1.18 | 1.24 |
| enrichment fold (if given as the odds ratio) | 1.34 | 1.90 | 1.30 | 1.41 |
| enrichment p-value (Fisher exact test) | 1.62E−03 | 1.47E−10 | 7.38E−04 | 1.27E−05 |

Thus, the results confirm that percentage of DE genes, DE gene fold change and associated significance decreases as the distance (number of layer/step away) from the perturbed target increases, and that DE genes are enriched in the downstream steps of HMGCR compared to non-DE genes. Taken together, the HMGCR-inhibition experiment validates the predictive networks and their topological structure.

Functional Validation

In other embodiments of the invention, the systems and methods perform validation of the prioritized KDs—HMGCR, FDPS and SQLE—in processes associated with insulin sensitivity and in particular, to insulin mediated glucose uptake in relevant metabolic cell types such as adipocytes and SKMCs. To that end, the Simpson-Golabi-Behmel syndrome (SGBS) human preadipocyte line and the human SKMC line HMCL-7304 were differentiated to terminally differentiated adipocytes and myotubes, respectively. In certain embodiments, validation efforts were focused on HMGCR, FDPS and SQLE as these three key drivers participate in the same metabolic pathway-cholesterol biosynthesis- and, in addition, statins (HMGCR inhibitors) are at the center of an intense debate about their effect on insulin resistance and type II diabetes risk.

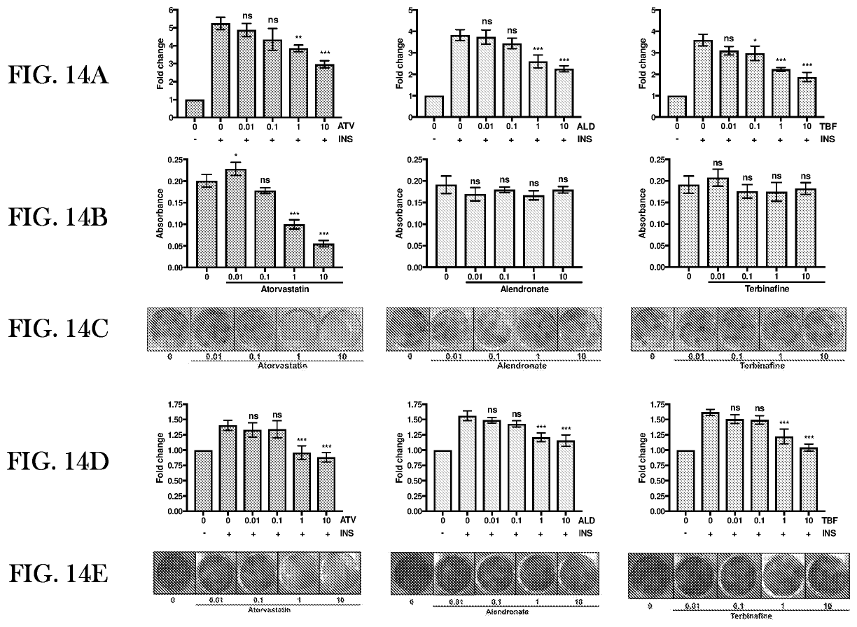

To functionally inhibit the three candidate genes, well-described and widely used chemical inhibitors are applied: atorvastatin (targeting HMGCR), alendronate (for FDPS) and terbinafine (for SQLE). As shown in FIG. 14A, all three inhibitors decrease insulin mediated glucose uptake in human adipocytes. However, only HMGCR inhibition demonstrates a detectable decrease of preadipocyte growth and differentiation efficiency (FIG. 14B, FIG. 14C). Along the same lines, atorvastatin inhibits both insulin mediated glucose uptake and cell proliferation in SKMCs, while alendronate and terbinafine show only a significant effect on glucose uptake (FIG. 14D, FIG. 14E). The results suggest that data driven co-expression and predictive networks combined with key driver analyses are powerful tools for the discovery of novel genes involved in IR.

Although GWAS studies have targeted T2D and insulin resistance- associated glycemic traits, the success in identifying new genes that contribute to insulin resistance risk has been fairly limited. The group has previously demonstrated that GWAS studies based on gold-standard measurements allows the discovery of novel genes associated with insulin resistance⁶ but the power to detect novel loci associated to IR has been limited by sample size. In addition, the genetic complexity and multicellular targets of insulin resistance advocate for the development of new cellular systems and holistic genetic approaches.

With this goal in mind, an iPSC library was generated with accurate measurements of insulin sensitivity that reflect the broad spectrum of insulin responses in human populations. Although the sample size is limited (52 IR vs. 48 IS individuals for a total of approximately 300 iPSC lines), differential gene expression analyses highlighted an enrichment for molecular pathways that are directly associated with insulin and glucose metabolism, suggesting that iPSC lines do reflect the insulin resistance status of the individuals they are derived from.

To overcome the sample size limitation and to develop a more holistic view of the genetic networks associated to IR, co-expression networks were built in certain embodiments for both IR and IS iPSC lines separately. In addition, for network construction, a dual approach was performed where the gene expression values for all samples were used (AS) (to increase power) or the average per patient (ApP) (to increase stringency). The constructed networks highlighted co-expression modules enriched for cellular functions like respiratory electron transport chain, glycolysis, cholesterol and steroid biosynthesis and glucose metabolism that are intimately associated with insulin sensitivity associated processes and the pathway enrichment signature arising from the DE analyses. Predictive network and key driver analyses were also performed to investigate the central genetic nodes that control the aforementioned modules and functions and thus, are most likely to be involved in the etiology of insulin resistance.

To better delimit and rank the key driver list, in certain embodiments, only the KDs defined were considered as such in both AS and ApP approaches (45 genes) and then the total number of appearances in the 4 constructed networks was considered (AS IR, AS IS, ApP IR and ApP IS), which rendered 9 top key drivers. As highlighted in Table 3, IDH1, BNIP3 and SLC27A1 (FATP-1) have been shown to participate in functions associated with insulin sensitivity or have been directly associated to insulin resistance or type 2 diabetes. Among the rest of the selected key drivers, the top two key drivers with the highest DE path and KD path values, which represents the connectivity of a given KD to DE genes and to other KDs, are Farnesyl Diphosphate Synthase (FDPS) and 3-Hydroxy-3-Methylglutaryl-CoA Reductase (HMGCR) which coordinately participate in the cholesterol biosynthetic pathway. Moreover, another gene participating in this pathway, SQLE is among the 45 KDs shared between both approaches. Meta-analysis of clinical trials with statins (HMGCR inhibitors) have shown an increase in T2D incidence that affects insulin resistant individuals in a disproportionate way. In addition, alleles in HMGCR that lower LDL-C confer an increased risk of developing T2D leading to speculation that statins affect insulin sensitivity or insulin secretion, although the exact cellular and molecular mechanisms to such an increase in T2D risk are still not well understood.

The predictive network not only illustrates co-regulated genes in the same pathway, but also demonstrates causality upstream and downstream of a given gene. There have been successful efforts to validate predictive networks, and therefore, it was useful to show that capture the downstream effector genes of key drivers in the predictive networks in certain embodiments of the invention. The empirical network validation through HMGCR inhibition demonstrated enrichment for DE genes and log fold change in the downstream proximity of HMGCR all act to validate the overall structure of the network.

The functional validation assays in human preadipocyte (SGBS) cells demonstrate that HMGCR inhibition decreases preadipocyte proliferation, and differentiation and insulin mediated glucose uptake in mature adipocytes, similar to previous results in mouse preadipocyte lines. In addition, both proliferation and insulin mediated glucose uptake are affected in the human SKMCs (HMCL-7304), treated with atorvastatin. FDPS and SQLE inhibition have also a significant effect decreasing insulin mediated glucose uptake in human adipocytes and myotubes, while not affecting proliferation or differentiation. These results suggest that the effects on proliferation and differentiation of adipocytes and SKMCs are independent of the effect on insulin mediated glucose uptake. In addition, SQLE inhibition exerts a comparable effect on insulin mediated glucose uptake when compared to HMGCR inhibition, which suggests that the underlying mechanisms could be mediated by the deregulation of intracellular or membrane bound cholesterol levels.

In summary, it can be concluded that: (1) iPSCs retain a donor-specific signature; (2) differential gene expression analyses between IR and IS iPSCs show an enrichment for pathways associated with insulin sensitivity; (3) IR iPSCs have a differential response to HMGCR inhibition when compared with IS cells and; (4) co-expression and predictive networks combined with key driver analyses uncover robust candidates to participate in IR. Taken together, these results demonstrate that iPSCs in accordance with the present invention offer a novel and sophisticated model for the study of IR and the associated cardiovascular disease, especially when relevant metabolic (adipocytes or SKMCs) and vascular (endothelium) cell types are generated from the iPSC library with accurate measurements of insulin sensitivity.

Patient Recruitment, Biological Parameters, Insulin Sensitivity Measurement

Patient recruitment includes blood sampling, and insulin sensitivity measurement was performed by a modified insulin suppression test in accordance with Knowles et al.

RNA-Seq Processing

STAR v2.4.0g1 was used to align RNA-seq reads to the human genome built GRCh37. Using featureCounts v1.4.4, the uniquely mapping reads overlapping genes was counted as annotated by ENSEMBL v70.

Statistical Analyses and Data Processing

Unless otherwise specified, statistical analyses and data processing steps were performed in this exemplary embodiment using R v3.0.3. One of ordinary skill in the art will readily understand that any analogous software may be substituted in other embodiments of the invention.

Expression Data Normalization and Covariate Adjustment

Log2 counts per million (CPM) and TMM normalization (as implemented in edgeR) were used in all analyses described in Example 2. Normalized counts were retained only for genes with over 1 CPM in at least 30% of the experiments, as discussed herein. As a final normalization of the gene counts, the voom function from the limma R package was used in certain embodiments of the invention.

All RNA-seq data analyses were performed on expression residuals corrected for the effects of technical (sequencing batches and RNA preparation kits, reprogramming source cell) and patient covariates (sex, ethnicity, age, BMI). Batch and RNA preparation kit were adjusted for as random effects using the variancePartition R library whereas reprogramming source cell, and patient characteristics were adjusted for as fixed effects using the limma package. Due to having multiple clones per patient, two sets of expression residuals were computed: one (AS) using all sample from all clones for every patient and one (ApP) where residuals per patient were averaged.

Genomic Data and Expression Quantitative Trait (eQTL) Analysis

Similar to those processing and analysis steps described above, eQTL analysis in accordance with the present invention was performed by filtering genotype data to remove markers with over 5% missing entries, minor allele frequency below 1% and Hardy-Weinberg p value<10⁻⁶. Genotypes were phased with SHAPEIT v2.r790, and missing genotypes were imputed with Impute2 v2.3.2 using the reference panel from the 1000 Genomes Project Phase 3. Markers with high imputation quality (INFO>0.5; and minor allele frequency over 1% were retained for downstream analyses.

Following standard practice, only individuals of European ancestry were included in the eQTL analysis in order to avoid false positives due to the correlation between ancestry and gene expression. Principal components analysis based on genome-wide genotype data identified 81 individuals of European ancestry for eQTL analysis. eQTL analysis was performed with MatrixEQTL v2.1.1 using the first 5 genotype principal components as covariates. Latent variables were identified in the gene expression data using PEER v1.0. Expression residuals were computed by removing the first 20 PEER components. Since multiple iPSC lines were assayed from each individual, the expression value for each individual was summarized as the mean expression residual value from the multiple lines for a given individual and gene. The mean values for each individual were subsequently quantile normalized for each gene. Cis-eQTL analysis considered markers within 1Mb of the transcription state site of each gene. False discovery rates were computed following Benjamini-Hochberg.”

Differential Expression Analysis

For the initial differential expression analysis, after alignment and feature counting, RNA-seq counts were normalized and residual expression was computed after adjusting for both technical (sequencing batch, RNA extraction method; modeled as random effects) and biological/population (reprogramming source cell, sex, ethnicity, age, BMI; modeled as fixed effects) covariates. the dataset was then split into IS and IR groups of individuals and adjusted, using a linear model, for patient ID in each group separately, but then adding the estimated intercept from the model back to the residual expression values. The two sets of residuals were then combined and DE analysis was performed. Since the intercept is an estimated parameter, even if the true population values were identical in the two groups, it was estimated that there would be slightly different numerical values since the dataset was finite, meaning that in this DE analysis, all genes would be significant. However, the hypothesis is that the difference in average expression due to insulin sensitivity status is dominating the random differences due to the intercept estimation procedure.

For the expression residuals used for the co-expression and predictive network analyses, 2 analysis streams were adopted: one where there was use of all samples without adjusting for patient ID (AS) and one where there was an averaging of residuals per patient (ApP). Differentially expressed genes were determined using a linear model, as implemented in the 1mFit function from the limma package v3.18.13 in R. Statistical significance was assessed using a cut-off of 0.05 on FDR adjusted p-values.

Co-Expression Networks and Selection of Co-Expression Modules

Co-expression networks were constructed in the exemplary embodiment using the coexpp R package, which provides an optimized workflow for the WGCNA R package (v1.14-1 used here together with R v3.0.3) for large numbers of genes. Seeding genes for the predictive network (specifically, the input to pathfinder) were selected to be the genes in co-expression network modules statistically enriched (FDR<0.05) for GO terms relevant to insulin resistance related traits (biological processes only).

Predictive Networks

The top-down and bottom-up predictive network pipeline was developed in the present invention to build causal predictive network models, which leverages the bottom-up belief propagation engine as a sub-routine to infer causality. The conventional (top-down) Bayesian networks cannot capture opposite causality, since this approach cannot distinguish between equivalent causalities. The bottom-up method leverages the nonlinearity of biochemical reactions to infer causality of a molecular interaction, i.e. fitting better to the data along true causal direction than false causal direction, thereby breaking the statistical equivalence. By integrating the novel bottom-up causality inference approach with (top-down) Bayesian networks, the integrated top-down & bottom-up predictive network platform will result in a complete causal network with causality resolved among equivalent structures. This pipeline inherits the advantage of BN in integrating the multi-scale ‘omics-’ data (genotype and transcriptomics) to construct multi-scale network models. The genotype data is incorporated as cis-eQTL genes in the model where they are constrained to be the top node (without other parents). We used the genes in the selected modules from co-expression networks as seeding genes for predictive network modeling.

Key Driver Analysis and Prioritization of Top Hits

Key Driver Analysis was performed using a modified version of the R package KDA (https://github.com/zhukuixi/KDA) in this particular embodiment of the invention. First, a background sub-network is defined by KDA by looking for a K-step upstream neighborhood round each node in the target gene list in the network. Second, starting from each node in this sub-network, KDA evaluates the enrichment of downstream neighborhoods (for each step size from 1 to K) for the target gene list. K=6 was used. The overlap of key drivers identified was taken from the AS and ApP networks and further ranked the top 9 KDs (described exemplarily in Table 3).

HMGCR Inhibition in iPSC Lines

iPSCs from 6 IS and 6 IR individuals were maintained in feeder-free conditions using mTesr1 (Stem Cell technologies, Inc) supplemented with 1 mM L-Glutamine, 1 mM Penicillin-Streptomycin, 0.1 μ/ml Fungizone.) on 5% matrigel coated 6-well TC plates. For passaging, cells were washed once with PBS and treated with pre-warmed 1 mM EDTA (Sigma), incubated at 37 degrees for 1-5 minutes, and resuspended in fresh mTesr1 medium 2 μM Thiazovivin (Millipore). After 12 hours incubation in Thiazovivin, medium was changed daily with fresh mTesr1. Cells were grown to ˜90-100% confluency, washed once with PBS and were treated with either DMSO (D) or 1 uM Atorvastatin (A) (Selleckhem) for 12 h. Cells were washed once in PBS and harvested for RNA extraction using PureLInk RNA mini kit (Thermo Fisher Scientific). Total RNA was quantified using a Nanodrop (Thermo Scientific). RNA samples with a A260/280 ratio <1.8 or >2.3 were excluded from further processing and the RNA was sequenced using the Illumina HiSeq 2500 system.

Cell Culture

Simpson-Golabi-Behmel syndrome (SGBS) cells (human preadipocytes) were provided by Dr. Martin Wabitsch (Ulm University, Ulm, Germany) SGBS cells were cultured in DMEM/F12 supplemented with 10% FBS, 33 uM biotin, and 17 uM panthotenate. The SKMC line HMCL-7304 cells were provided by Institute of Child Health (ICH), University College London. Cells were cultured in SKMC growth medium (PromoCell). iPSCs were generated and cultured as described above.

Adipogenic and Skeletal Muscle Differentiation

For adipogenic differentiation SGBS cells were grown to confluence and subjected to a two-step differentiation process. Cells were first exposed for 3 days to media composed of DMEM/F12 supplemented with 0.01 mg/mL of transferrin, 20 uM of insulin, 100 nM cortisol, 0.2 nM 3,3′,5-Triiodo-L-thyronine, 25 nM dexamethasone, 250 uM 3-Isobutyl-1-methylaxanthine, and 2 uM rosiglitazone. Afterwards, cells were exposed to DMEM/F12 supplemented with 0.01 mg/mL of transferrin, 20 uM of insulin, 100 nM cortisol, and 0.2 nM 3,3′,5-Triiodo-L-thyronine for additional 12 days. HMCL-7304 cells were differentiated in presence of SKMC differentiation medium (PromoCell) for 4-5 days before glucose uptake was performed.

For quantification of the effect on adipogenic differentiation, chemical inhibitors for HMGCR (atorvastatin), FDPS (alendronate), and SQLE (terbinafine) were added at day 0 of differentiation at different concentrations (10 nM, 100 nM, 1 uM, 10 uM.). Differentiation quantification was performed with Oil Red 0. Differentiated SGBS cells were fixed with 10% formalin for 10 minutes at room temperature. After washing with 60% isopropanol, samples were incubated in Oil Red 0 (Sigma-Aldrich) for 10 minutes at room temperature. Oil Red 0 was eluted with 100% isopropanol for 10 minutes. The solution was then transferred into a 96-well plate and absorbance measured at 500nm.

Glucose Uptake

Differentiated SGBS or HMCL-7304 cells were pretreated for 24 hours with different concentrations (10 nM, 100 nM, 1 uM, 10 uM) of the chemical inhibitors for HMGCR (atorvastatin), FDPS (alendronate), and SQLE (terbinafine). After the preincubation, the cells were starved for 2 hours prior to 30 minutes of 100 nM insulin stimulation at 37 degrees. Following stimulation, cells were incubated with Krebs-Ringer bicarbonate-HEPES (KRBH) buffer (130 mM NaCl, 5 mM KCl, 1.3 mM CaCl₂, 1.3 mM MgSO₄, 25 mM HEPES, pH 7.4) containing 100 uM 2-deoxy-D-glucose, and 1 uCi/ml 2-deoxy-D-[1,2-³H]glucose for 10 minutes at room temperature. The cells were then washed with PBS, harvested in 300 uL of M-PER lysis buffer (ThermoFisher), and then added to scintillation vials containing 4.75 mL of scintillation fluid (Perk Elmer).

Radioactive counts were determined with a scintillation counter (Model ID: Beckman LS6500). Excess samples were subjected to BCA assay for protein quantification and normalization of radioactive counts. All samples were represented as fold change compared to the unstimulated (no insulin) condition.

Growth Assay

SGBS or HMCL-7304 cells were plated at 50 or 100 cells/cm2 in 12 well plates and were grown for 12 to 14 days in the absence or presence of 10 nM, 100 nM, 1 uM, 10 uM of atorvastatin, terbinafine or alendronate. After the treatment, the cells were fixed in cold methanol for 15 minutes and stained with crystal violet for 10 minutes. Dye excess was washed with water and pictures taken immediately afterwards.

The system and method of the present invention may be implemented by computer software that permits the accessing of data from an electronic information source. The software and the information in accordance with the invention may be within a single, free-standing computer or it may be in a central computer networked to a group of other computers or other electronic devices. The information may be stored on a computer hard drive, on a CD-ROM disk or on any other appropriate data storage device.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A computer-implemented method for predictive network modeling comprising the steps of:
aggregating computational data from one or more databases;
computing a multiscale network based on the computational data;
passing the multiscale network through Monte Carlo Markov Chain sampling;
computing a top-down causal model; and
computing a bottom-up predictive model,
wherein the top-down causal model is used to infer conditional independence among a plurality of variables and the bottom-up predictive model is used to infer causality among equivalent variable structures in the plurality of variables.

2. The method of claim 1, wherein the computational data originates from scientific databases.

3. The method of claim 1, wherein the Markov Chain Monte Carlo sampling is comprised of hill-climbing sampling, global sampling, and order-based sampling.

4. The method of claim 1, wherein the plurality of variables are associated with drug targets.

5. The method of claim 1, further comprising the step of updating the computed top-down causal model and the bottom-up predictive model by cycling said models through one or more iterations of Monte Carlo Markov Chain sampling.

6. A system for predictive network modeling comprised of:
one or more computers; and
a network;
wherein the system aggregates computational data from one or more databases, computes a multiscale network based on the computational data, passes the multiscale network through Monte Carlo Markov Chain sampling, computes a top-down causal model and a bottom-up predictive model, and wherein the top-down causal model is used to infer conditional independence among a plurality of variables and the bottom-up predictive model is used to infer causality among equivalent variable structures in the plurality of variables.

7. The system of claim 6, wherein the computational data originates from scientific databases.

8. The system of claim 6, wherein the Monte Carlo Markov Chain sampling is comprised of hill-climbing sampling, global sampling, and order-based sampling.

9. The system of claim 6, wherein the plurality of variables are associated with drug targets.

10. The system of claim 6, wherein the system updates the computed top-down causal model and the bottom-up predictive model by cycling said models through one or more iterations of Markov Chain Monte Carlo sampling.

11. A method of predictive modeling comprising the steps of:
extracting blood from one or more individuals;
compiling one or more biometric parameters from the blood;
reprogramming a plurality of induced pluripotent stem cells;
performing RNA sequencing of the induced pluripotent stem cells to obtain RNA sequencing data;
performing predictive network analysis on the RNA sequencing data to obtain predictive network analysis data; and
performing key driver analysis on the predictive network analysis data, wherein the key driver analysis is used to identify target gene candidates.

12. The method of claim 11, wherein the predictive network analysis incorporates prior network analysis data.

13. The method of claim 11, wherein the key driver analysis results in a ranking of target gene candidates.

14. The method of claim 11, further comprising the step of performing residual expression analysis on the RNA sequencing data to obtain residual expression data, wherein said residual expression data is used in the key driver analysis;

15. The method of claim 14, wherein the residual expression data us analyzed for all samples and as an average-per-patient.

16. The method of claim 11 further comprising the step of performing differential expression analysis on the RNA sequencing data to obtain differential expression data, wherein said differential expression data is used in the key driver analysis.

17. The method of claim 11, further comprising performing co-expression analysis on the RNA sequencing data to obtain co-expression data, wherein said co-expression data is used in the key driver analysis.

18. The method of 11, wherein the biometric parameters are age, body mass index, sex and race/ethnicity.

19. The method of claim 11, wherein the target gene candidates correspond to insulin sensitivity or insulin resistance.

20. The method of claim 11, wherein the induced pluripotent stem cells are clones.
